(19) United States
(12) Patent Application Publication
(10) Pub. No.: US 2012/0059415 A1
(43) Pub. Date: Mar. 8, 2012

(54) KNOTLESS SUTURE ANCHOR FOR SECURING SOFT TISSUE TO BONE

(76) Inventor: Joseph H. Sklar, Longmeadow, MA (US)

(21) Appl. No.: 13/228,231

(22) Filed: Sep. 8, 2011

Related U.S. Application Data
(63) Continuation-in-part of application No. 11/347,082, filed on Feb. 3, 2006.
Application No. 11/347,082 is a continuation-in-part of application No. 13/113,681, filed on May 23, 2011.
(60) Provisional application No. 60/650,759, filed on Feb. 7, 2005.
(60) Provisional application No. 61/346,954, filed on May 21, 2010.
(60) Provisional application No. 61/380,909, filed on Sep. 8, 2010.
(60) Provisional application No. 61/419,275, filed on Dec. 3, 2010.
(60) Provisional application No. 61/425,028, filed on Dec. 20, 2010.

(51) Int. Cl. A61B 17/04

(52) U.S. Cl. 606/232

(57) ABSTRACT

A suture anchor for anchoring a length of suture to bone, the suture anchor comprising:
an elongated expandable body having a distal end surface, a proximal end surface, an exterior sidewall extending between the distal end surface and the proximal end surface, and an interior passageway extending between the distal end surface and the proximal end surface; and
an actuator comprising a distal end, a proximal end and a shaft extending between the distal end and the proximal end, the distal end of the actuator comprising a suture capture mechanism movably disposed relative to the elongated body;
wherein the suture capture mechanism comprises a diamond-shaped structure configured to provide an opening into the interior thereof.

### REFERENCE TO PENDING PRIOR PATENT APPLICATIONS

This patent application:

(1) is a continuation-in-part of pending prior U.S. patent application Ser. No. 11/347,082, filed Feb. 3, 2006 by Joseph H. Sklar for KNOTLESS SUTURE ANCHOR (Attorney's Docket No. SKLAR-32A), which patent application in turn claims benefit of prior U.S. Provisional Patent Application Ser. No. 60/650,759, filed Feb. 7, 2005 by Joseph H. Sklar for KNOTLESS SUTURE ANCHOR (Attorney's Docket No. SKLAR-32A PROV);

(2) is a continuation-in-part of pending prior U.S. patent application Ser. No. 13/113,681, filed May 23, 2011 by Joseph H. Sklar for KNOTLESS SUTURE ANCHOR FOR SECURING SOFT TISSUE TO BONE (Attorney's Docket No. SKLAR-33), which in turn claims benefit of prior U.S. Provisional Patent Application Ser. No. 61/346,954, filed May 21, 2010 by Joseph H. Sklar for KNOTLESS SUTURE ANCHOR FOR SECURING SOFT TISSUE TO BONE (Attorney's Docket No. SKLAR-33 PROV);

(3) claims benefit of pending prior U.S. Provisional Patent Application Ser. No. 61/380,909, filed Sep. 8, 2010 by Joseph H. Sklar for KNOTLESS SUTURE ANCHOR FOR SECURING SOFT TISSUE TO BONE (Attorney's Docket No. SKLAR-34 PROV);

(4) claims benefit of pending prior U.S. Provisional Patent Application Ser. No. 61/419,275, filed Dec. 3, 2010 by Joseph H. Sklar for KNOTLESS SUTURE ANCHOR FOR SECURING SOFT TISSUE TO BONE (Attorney's Docket No. SKLAR-35 PROV); and

(5) claims benefit of pending prior U.S. Provisional Patent Application Ser. No. 61/425,028, filed Dec. 20, 2010 by Joseph H. Sklar for KNOTLESS SUTURE ANCHOR FOR SECURING SOFT TISSUE TO BONE (Attorney's Docket No. SKLAR-36 PROV).

The seven above-identified patent applications are hereby incorporated herein by reference.

### FIELD OF THE INVENTION

This invention relates to surgical devices in general, and more particularly to suture anchors of the sort adapted to anchor a segment of suture in bone, such that other segments of the suture can be used to secure soft tissue to bone.

### BACKGROUND OF THE INVENTION

Numerous devices are currently available to attach objects to bone. More particularly, screws, staples, cement and sutures have all been used to attach soft tissue (e.g., ligaments, tendons, muscles, etc.), bone and inanimate objects (e.g., prostheses) to bone.

In certain situations it can be desirable to anchor a segment of suture in the bone, such that other segments of the suture can be used to attach a desired object (e.g., a ligament or prosthesis) to the bone. This is traditionally accomplished by first forming a hole in the host bone, then securing a segment of suture to a suture anchor, and then securing the suture anchor in the hole in the bone, with the suture anchor securing the suture to the host bone. Other segments of the suture can then be used to fasten the desired object to the bone. Among other things, such suture anchors have found widespread application in procedures for re-attaching ligaments to bone, e.g., to restore a torn rotator cuff in the shoulder.

Traditionally, suture anchors have anchored an intermediate segment of the suture in the host bone, leaving the two free ends of the suture to secure the object (e.g., a piece of soft tissue such as ligament) to the bone. Such attachment may be effected by passing one or both of the two free ends of the suture through the soft tissue, or by passing one or both of the two free ends of the suture over the soft tissue, and then knotting the two free ends of the suture so as to effectively tie the object to the bone.

While such suture anchors have proven highly effective, it can sometimes be difficult and/or inconvenient to knot the suture at the surgical site where access to the surgical site is limited, e.g., such as in the case of an arthroscopic procedure. In situations such as these, it is common for the suture knot to be formed by (i) forming a first suture throw in the two free ends of the suture in the region outside the body, (ii) running the first suture throw down the two suture ends so as to engage the soft tissue at the interior surgical site, (iii) forming a second suture throw in the two free ends of the suture in the region outside the body, (iv) running the second suture throw down the two suture ends so as to engage the first suture throw disposed at the interior surgical site, and then (v) continuing the aforementioned procedure as many times as may be necessary in order to form a stable knot. This process can be difficult to accomplish, particularly where the knot must maintain proper tension against the soft tissue, and in any case it tends to be tedious and time-consuming.

Furthermore, in some surgeries there may be a substantial number of suture segments disposed at the surgical site, and it can be difficult to efficiently manage those suture segments at the surgical site, particularly where those suture segments may need to be manipulated about the interior surgical site and/or advanced to, or removed from, the interior surgical site.

In addition to the foregoing, it should also be appreciated that the knots used to tie down soft tissue to the bone can present additional complications. By way of example but not limitation, overlying tissue can impinge upon the knots, causing trauma to the tissue and in many cases presenting substantial pain to the patient, particularly when the knots are large. This problem may be exacerbated where the knots are placed close to articulating anatomy, such as the moving bones and/or other tissue of a joint.

As a result, one object of the present invention is to provide a new and improved suture anchor which can be used to attach an object to bone without requiring that a knot be tied.

And another object of the present invention is to provide a new and improved method for attaching an object to bone without requiring that a knot be tied.

### SUMMARY OF THE INVENTION

These and other objects of the present invention are achieved through the provision and use of a novel suture anchor for anchoring a length of suture to bone.

In one form of the invention, there is provided a suture anchor for anchoring a length of suture to bone, the suture anchor comprising:

an elongated body having a distal end surface, a proximal end surface, an exterior sidewall extending between the distal end surface and the proximal end surface, and an interior passageway extending between the distal end surface and the proximal end surface; and

an actuator comprising a distal end, a proximal end and a shaft extending between the distal end and the proximal end, the distal end of the actuator comprising a suture capture mechanism, the proximal end being configured for engagement by a handle, and the shaft being slidably disposed within the passageway;

the elongated body and the actuator being configured so that:
(i) when the actuator is in a first, extended position relative to the elongated body, the suture capture mechanism is spaced a sufficient distance from the distal end surface of the elongated body such that a length of suture may enter the suture capture mechanism;
(ii) when the actuator is in a second, intermediate position, the suture capture mechanism is positioned relative to the distal end surface of the elongated body such that the suture is slidably captured within the suture capture mechanism of the elongated body; and
(iii) when the actuator is in a third, retracted position relative to the elongated body, the suture capture mechanism is positioned relative to the distal end surface of the elongated body such that the suture is securely captured within the suture capture mechanism of the elongated body.

In another form of the invention, there is provided a method for securing a suture to a bone, comprising:

providing a suture anchor comprising an elongated body and a suture capture mechanism slidably disposed relative to the elongated body:

positioning the suture capture mechanism so that it is spaced from the elongated body;

passing a suture into the suture capture mechanism;

repositioning the suture capture mechanism relative to the elongated body so that the suture is slidably captured within the suture capture mechanism of the elongated body;

positioning the elongated body in a bone hole; and

repositioning the suture capture mechanism relative to the elongated body so that the suture is bound within the suture capture mechanism of the elongated body, with the elongated body being secured in the bone hole.

In another form of the invention, there is provided a suture anchor for anchoring a length of suture to bone, the suture anchor comprising:

an elongated expandable body having a distal end surface, a proximal end surface, an exterior sidewall extending between the distal end surface and the proximal end surface, and an interior passageway extending between the distal end surface and the proximal end surface; and

an actuator comprising a distal end, a proximal end and a shaft extending between the distal end and the proximal end, the distal end of the actuator comprising a suture capture mechanism movably disposed relative to the elongated body;

wherein the suture capture mechanism comprises a diamond-shaped structure configured to provide an opening into the interior thereof.

In another form of the invention, there is provided a method for anchoring a length of suture to bone, the method comprising:

providing a suture anchor comprising:
an elongated expandable body having a distal end surface, a proximal end surface, an exterior sidewall extending between the distal end surface and the proximal end surface, and an interior passageway extending between the distal end surface and the proximal end surface; and
an actuator comprising a distal end, a proximal end and a shaft extending between the distal end and the proximal end, the distal end of the actuator comprising a suture capture mechanism movably disposed relative to the elongated body;
wherein the suture capture mechanism comprises a diamond-shaped structure configured to provide an opening into the interior thereof;

passing a suture into the suture capture mechanism;

repositioning the suture capture mechanism relative to the elongated body.

### BRIEF DESCRIPTION OF THE DRAWINGS

These and other objects and features of the present invention will be more fully disclosed or rendered obvious by the following detailed description of the preferred embodiments of the invention, which are intended to be read in conjunction with the accompanying drawings, wherein like numbers refer to like parts, and further wherein:

FIG. 1 is a schematic view showing one preferred form of suture anchor formed in accordance with the present invention;

FIG. 2 is a view like that of FIG. 1, except showing the suture anchor in exploded view;

FIG. 3 is a cross-sectional view showing the suture anchor with its actuator in its first, extended position;

FIG. 4 is a schematic side view taken along line 4-4 of FIG. 3;

FIG. 5 is a cross-sectional view showing the suture anchor with its actuator in its second, intermediate position;

FIG. 6 is a schematic side view taken along line 6-6 of FIG. 5;

FIG. 7 is a cross-sectional view showing the suture anchor with its actuator in its third, retracted position;

FIG. 8 is a schematic side view taken along line 8-8 of FIG. 7; and

FIGS. 9-16 are a series of schematic views showing soft tissue being attached to bone using the suture anchor shown in FIGS. 1-8 (with FIGS. 14 and 16 being sectional views taken along lines 14-14 and 16-16 of FIGS. 13 and 15, respectively;

FIG. 17 is a schematic view showing another preferred form of suture anchor formed in accordance with the present invention;

FIG. 18-21 are schematic views showing various components of the suture anchor shown in FIG. 17;

FIGS. 22-30 are schematic views showing various dispositions of the suture anchor shown in FIG. 17;

FIGS. 31-33 are schematic views showing suture being grappled by the suture anchor shown in FIG. 17 and then secured to bone;

FIGS. 34-49 are schematic views showing an additional suture anchor formed in accordance with the present invention;

FIGS. 50-69 are a series of schematic views showing soft tissue being attached to bone using the suture anchor shown in FIGS. 34-49;

FIG. 70 is a schematic view showing another preferred form of suture anchor formed in accordance with the present invention;

FIGS. 71-73 are cross-sectional views showing the suture anchor of FIG. 70 with its actuator in its first, extended position;

FIGS. 74 and 75 are cross-sectional views showing the suture anchor of FIG. 70 with its actuator in its second, intermediate position; and

FIGS. 76-78 are cross-sectional views showing the suture anchor of FIG. 70 with its actuator in its third, retracted position.

### DETAILED DESCRIPTION OF THE PREFERRED EMBODIMENTS

### “Expandable Body” Construction

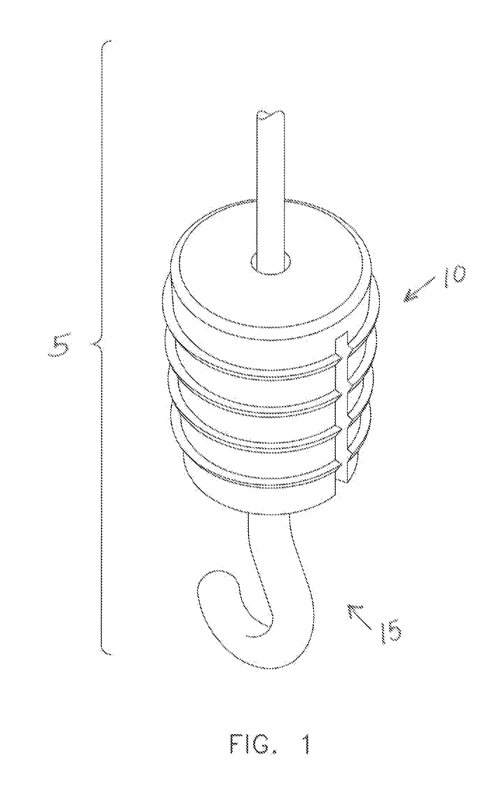

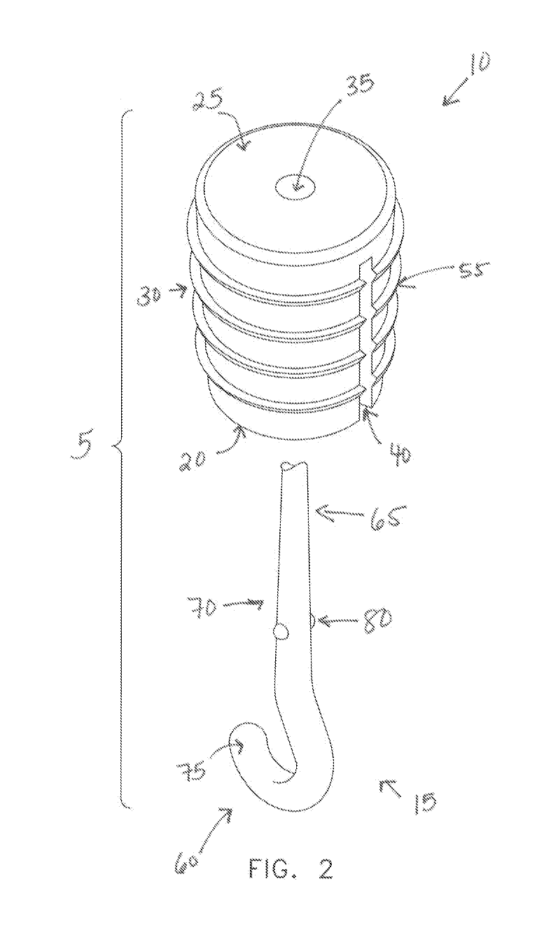

Looking first at FIGS. 1 and 2, there is shown a suture anchor 5 for anchoring a length of suture to bone, such that the suture can thereafter be used to attach an object (e.g., a ligament) to the bone. In accordance with the present invention, suture anchor 5 generally comprises an elongated body 10 and an actuator 15.

Elongated body 10 comprises a distal end surface 20, a proximal end surface 25, an exterior sidewall 30 extending between distal end surface 20 and proximal end surface 25, and an interior passageway 35 extending between distal end surface 20 and proximal end surface 25. A pair of diametrically-opposed side slots 40 extend between exterior sidewall 30 and interior passageway 35.

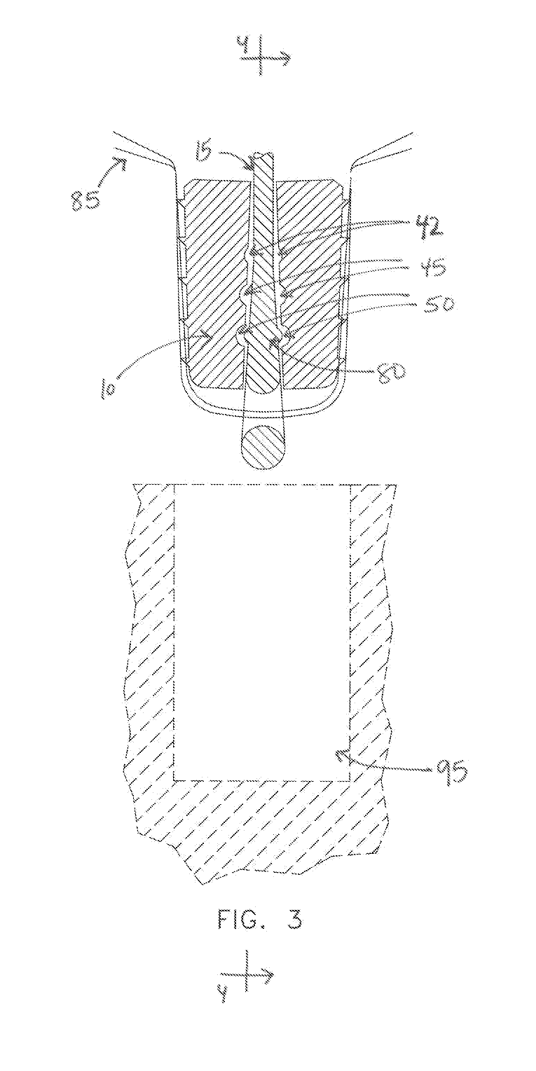

Elongated body 10 has a generally cylindrical configuration, but is formed out of a flexible or otherwise expandable material, such that elongated body 10 can have its cross-sectional profile changed in accordance with the longitudinal position of actuator 15 relative to elongated body 10, as will hereinafter be discussed in further detail. To this end, interior passageway 35 of the elongated body is preferably tapered along its longitudinal length (FIG. 3), expanding in diameter as it extends distally, such that the actuator can essentially cam the elongated body open as the actuator is moved proximally relative to the elongated body, as will hereinafter be discussed in further detail. In order to regulate the disposition of actuator 15 relative to elongated body 10, interior passageway 35 comprises three pairs of detents, 42, 45 and 50 (see FIG. 3), which releasably engage actuator 15, as will hereinafter be discussed in further detail.

The outer surface 30 of elongated body 10 may comprise ribbing 55 (FIG. 2) or roughening along a portion thereof so as to assist in securing a suture between exterior sidewall 30 of elongated body 10 and a wall of a bone hole and/or to assist in securing elongated body 10 to a wall of a bone hole.

Actuator 15 comprises a distal end 60, a proximal end 65 and a shaft 70 extending between distal end 60 and proximal end 65. Distal end 60 of actuator 65 comprises a J-hook 75, proximal end 65 of actuator 15 is configured for engagement by a handle (not shown), and shaft 70 is configured to be slidably disposed within interior passageway 35 of elongated body 10. In order to regulate the disposition of actuator 15 relative to elongated body 10, shaft 70 comprises a pair of projections 80, which releasably engage detents 42, 45 and 50 of interior passageway 35, as will hereinafter be discussed in further detail.

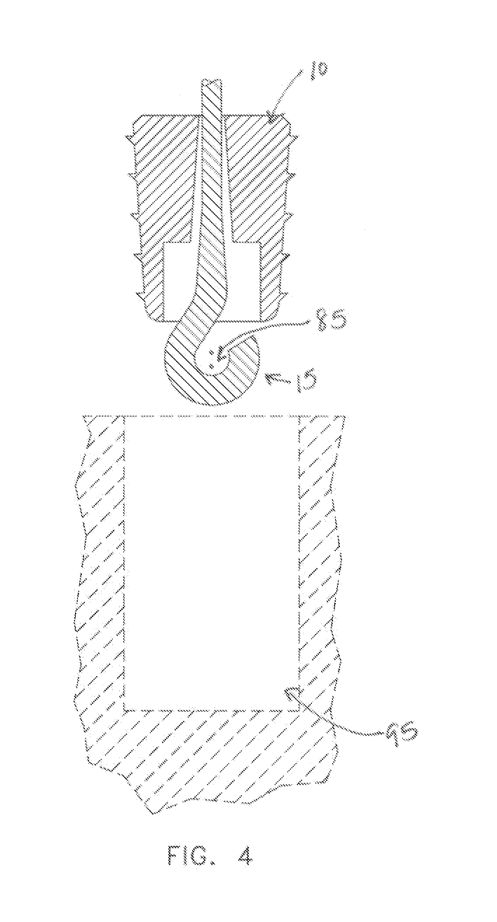

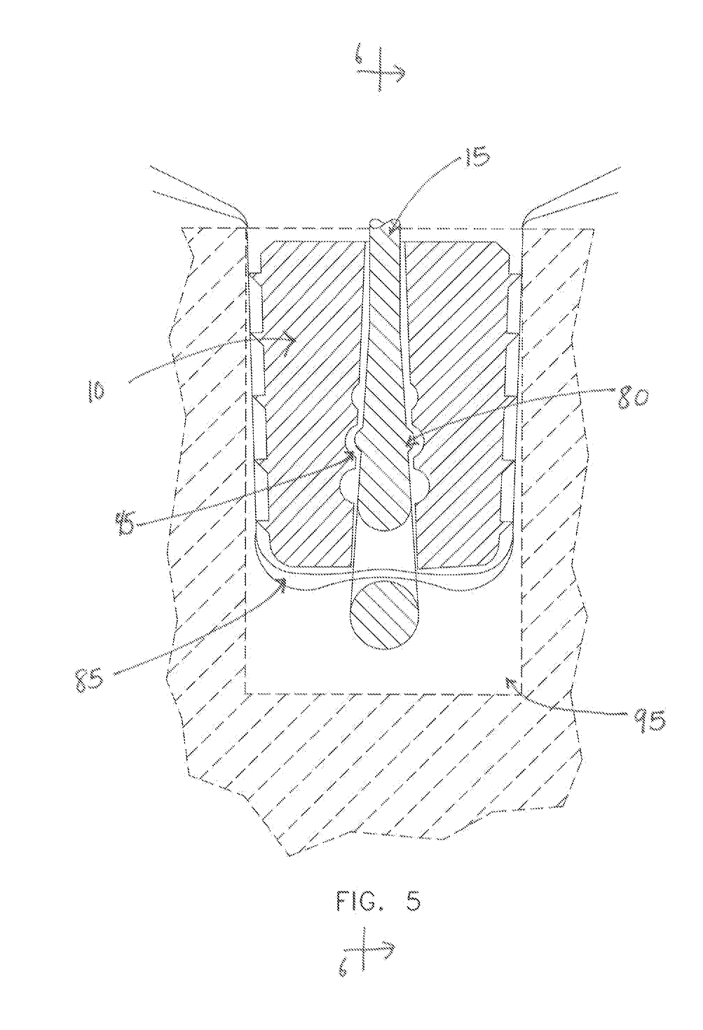

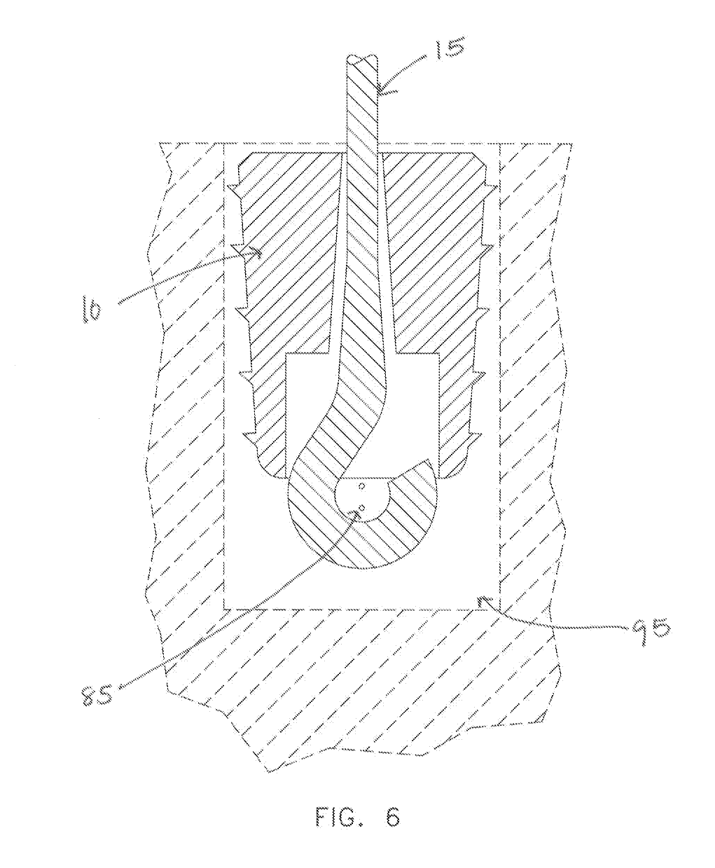

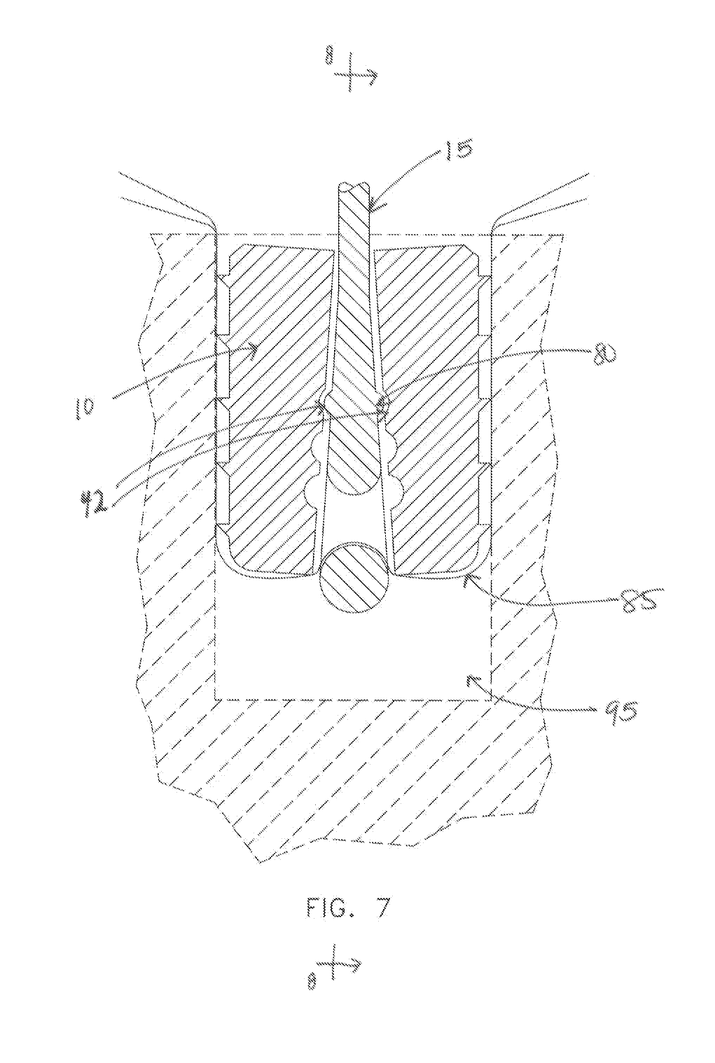

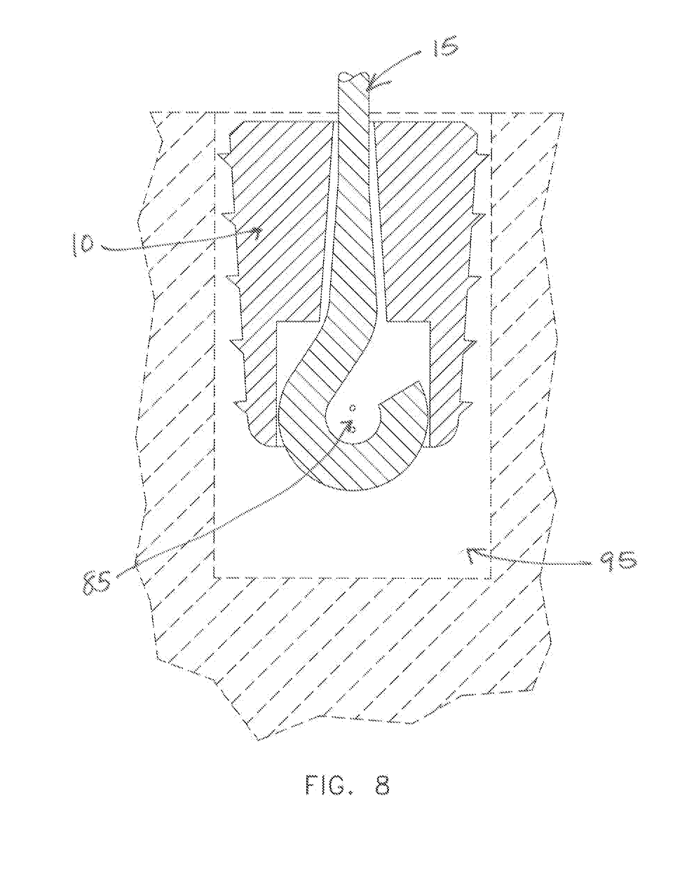

Looking next at FIGS. 3-8, elongated body 10 and actuator 15 are configured so that actuator 15 may be disposed between (i) a first, extended position (FIGS. 3 and 4, (ii) a second, intermediate position (FIGS. 5 and 6), and (iii) a third, retracted position (FIGS. 7 and 8), in order that the suture anchor may successively grapple, manipulate and, ultimately, anchor a length of suture to a bone.

More particularly, and looking at FIGS. 3 and 4, when actuator 15 is in a first, extended position relative to elongated body 10, elongated body 10 has a first cross-sectional profile and J-hook 75 is spaced a sufficient distance from distal end surface 20 of elongated body 10 such that J-hook 75 may pick up (e.g., grapple) a length of suture 85. More particularly, in the first extended position, elongated body 10 has a generally cylindrical, but preferably also slightly tapered, configuration so that suture anchor 5 may be easily inserted into a bone hole after J-hook 75 picks up (e.g., grapples) a length of suture.

Furthermore, as shown in FIG. 3, when actuator 15 is in its first, extended position relative to elongated body 10, projections 80 of actuator 15 are releasably engaged with detents 50 of interior passageway 35. As a result, J-hook 75 is maintained in position relative to distal end surface 20 of elongated body 10.

Looking now at FIGS. 5 and 6, when actuator 15 is in a second, intermediate position, elongated body 10 substantially retains its first cross-sectional profile and J-hook 75 is positioned relative to distal end surface 20 of elongated body 10 such that a suture 85 is slidably captured between J-hook 75 and distal end surface 20 of elongated body 10. More particularly, in the second, intermediate position, elongated body 10 has a generally cylindrical, but slightly tapered configuration, so that suture anchor 5 may be inserted into a bone hole 95 after J-hook 75 picks up a length of suture 85. Furthermore, the length of suture is slidably captured between J-hook 75 and distal end surface 20 of elongated body 10 so that the length of suture can still be moved relative to elongated body 10 as suture anchor 5 is inserted into a bone hole.

Furthermore, as shown in FIG. 5, when actuator 15 is in its second, intermediate position relative to elongated body 10, projections 80 of actuator 15 are releasably engaged with detents 45 of interior passageway 35. As a result, J-hook 75 is maintained in position relative to distal end surface 20 of elongated body 10.

Looking now at FIGS. 7 and 8, when actuator 15 is in a third, retracted position relative to elongated body 10, elongated body 10 has a second, expanded cross-sectional profile and suture 85 is securely captured between J-hook 75 and distal end surface 20 of elongated body 10. More particularly, in the third, retracted position, elongated body 10 has been expanded diametrically (expanding diametrically-opposed side slots 40 in the process) by the movement of projections 80 of actuator 15 into detents 40 of interior passageway 35.

Furthermore, by moving actuator 15 into its third, retracted position, the length of suture grappled by J-hook 75 is securely captured inside interior passageway 35 of elongated body 10 so that the suture is secured at distal end surface 20 of elongated body 10, e.g., where J-hook 75 enters interior passageway 35. In this respect it will be appreciated that the size and configuration of J-hook 75 is coordinated with the size and configuration of the diametrically-expanded elongated body 10 (including the size and configuration of the expanded diametrically-opposed side slots 40) so as to ensure that the suture is secured to elongated body 10 when actuator 15 is in its third, retracted position.

In addition, with the diametrical expansion of elongated body 10, elongated body 10 is secured within bone hole 95 and the length of suture is further captured between exterior sidewall 30 of elongated body 10 and a side wall of bone hole 95.

Thus it will be seen that, when suture anchor 5 is fully deployed within bone hole 95, three different types of fastening will occur: (i) elongated body 10 will be secured to the side wall of bone hole 95 due to the cross-sectional expansion of elongated body 10, (ii) suture 85 will be bound to elongated body 10 due to the retraction of actuator 15 into the interior of elongated body 10, and (iii) suture 85 will be pinched between the expanded elongated body 10 and the side wall of bone hole 95.

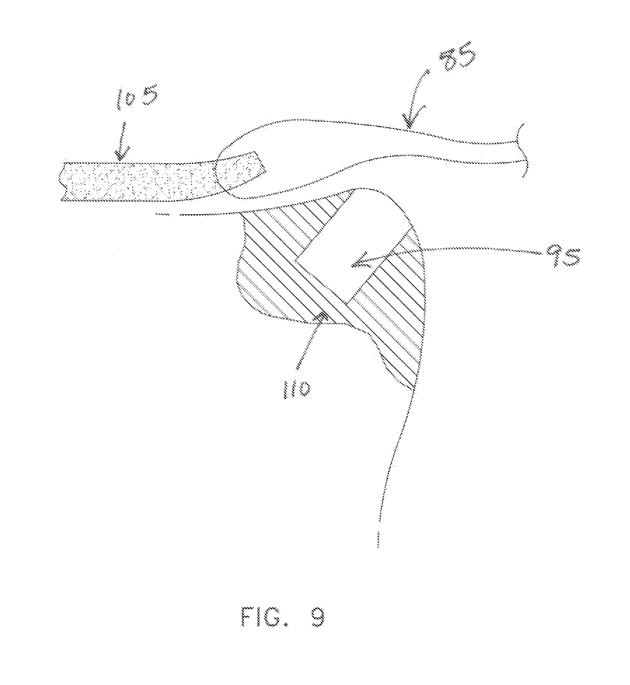

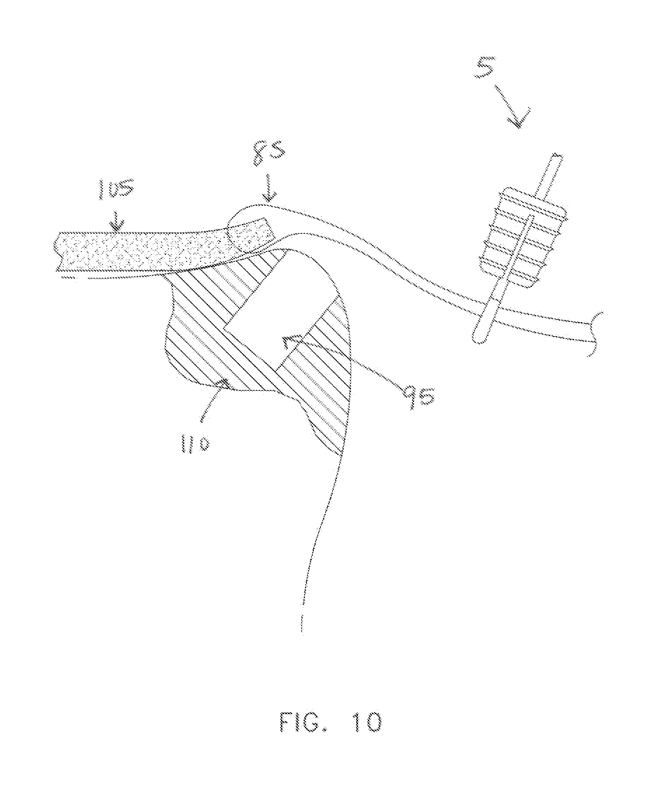

In use, and looking first at FIG. 9, bone hole 95 is first formed in a host bone 110, suture anchor 5 is positioned in its first, extended position (i.e., the position shown in FIGS. 3 and 4), suture 85 is passed through an object 105 (e.g., a rotator cuff) which is to be attached to bone 110, the two free ends of suture 85 are placed parallel to one another and tensioned (e.g., so as to pull object 105 into position relative to bone 110) and then, while tension is maintained on suture 85, J-hook 75 is used to hook (e.g., grapple) suture 85 (FIG. 10).

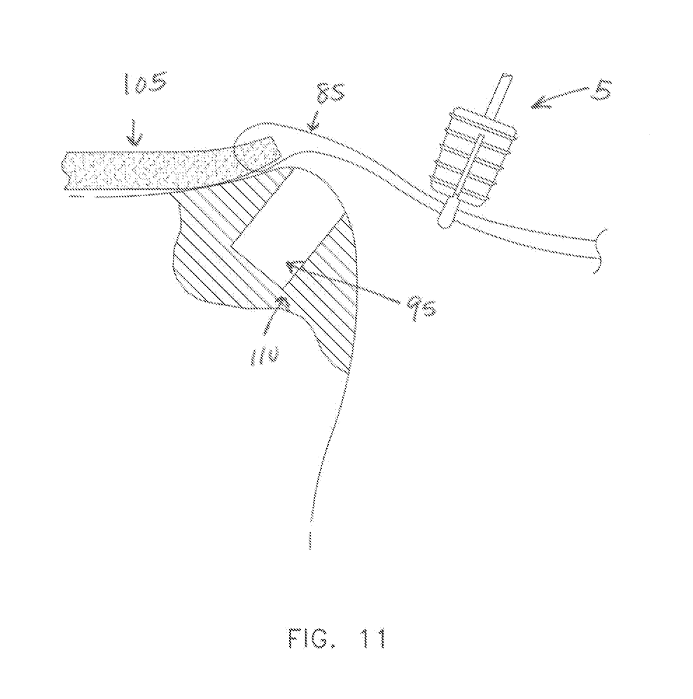

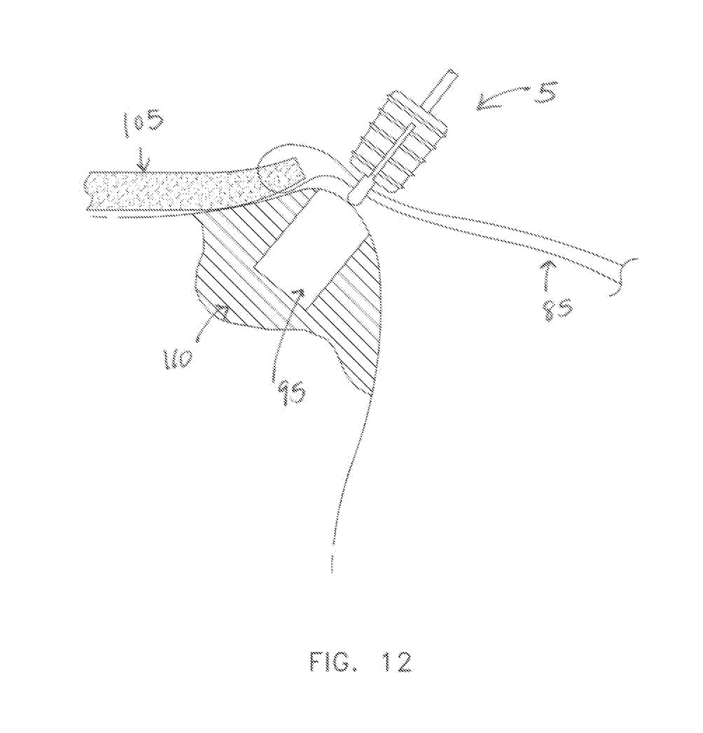

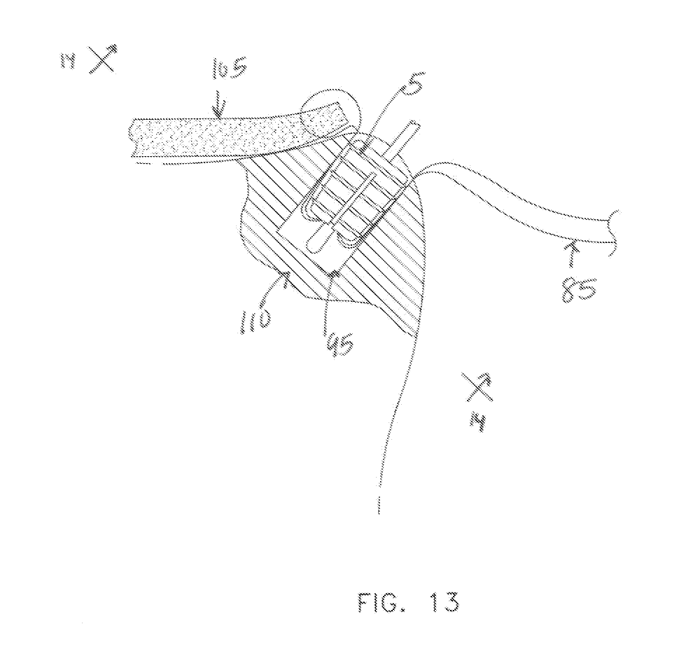

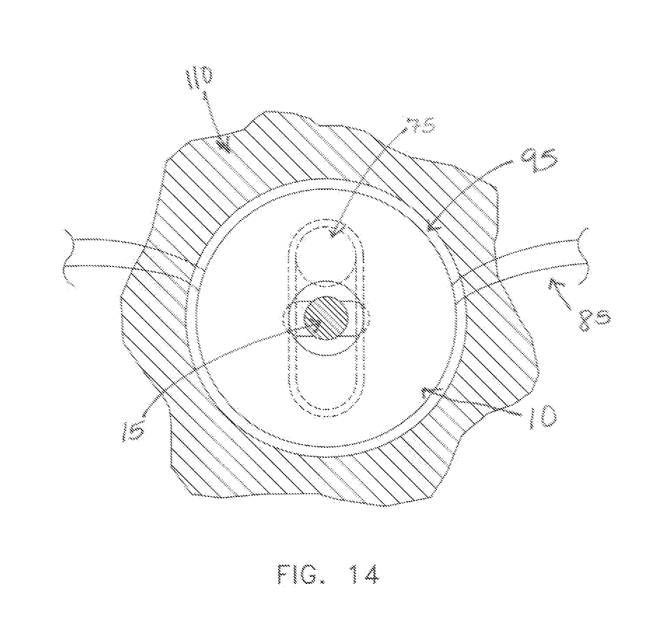

Next, and looking now at FIG. 11, actuator 15 of suture anchor 5 is moved from its first, extended position to its second, intermediate position (i.e., the position shown in FIGS. 5 and 6) so that suture 85 is slidably captured between J-hook 75 and distal end surface 20 of elongated body 10. Then elongated body 10 is inserted into bone hole 95, with suture 85 extending alongside exterior sidewall 30 of elongated body 10 (FIGS. 11-14). In this respect it will be appreciated that suture anchor 5 will slip easily into bone hole 95, since elongated body 10 of suture anchor 5 preferably has a generally tapered configuration and elongated body 10 has not yet been diametrically expanded.

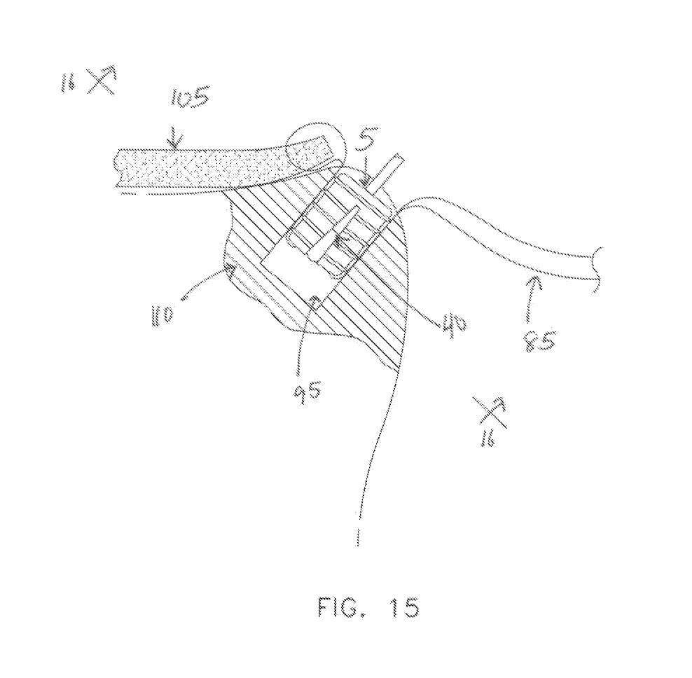

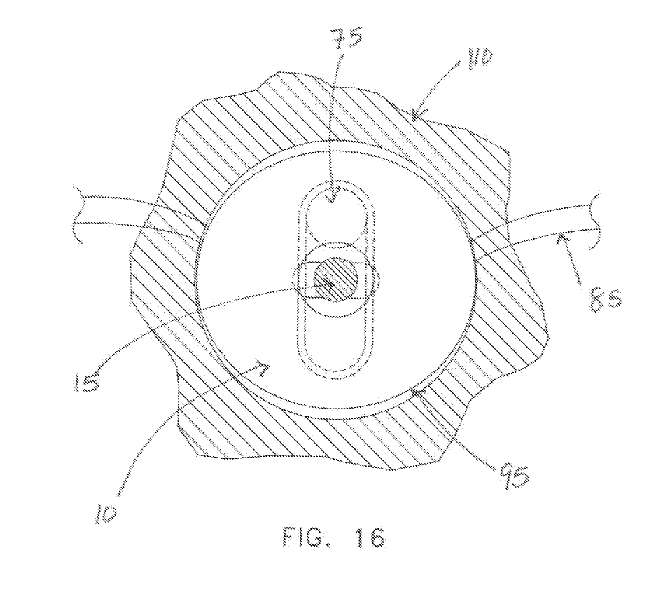

After elongated body 10 has been seated in bone hole 95, and looking now at FIGS. 15 and 16, shaft 70 of actuator 15 is moved from its second, intermediate position to its third, retracted position (i.e., the position shown in FIGS. 7 and 8), whereby to diametrically expand elongated body 10 (expanding diametrically-opposed slots 40 in the process) so that suture 85 is securely captured between J-hook 75 and distal end surface 20 of elongated body 10, elongated body 10 is secured to the bone, and suture 85 is further captured between exterior sidewall 30 of elongated body 10 and the side wall of bone hole 95, whereby to capture suture 85 (and hence object 105) to the bone.

In this way object 105 may be secured to bone 110 without the necessity of tying knots in suture 85.

In connection with the foregoing, it should be appreciated that J-hook 75 may be used to grapple the suture and slidably capture the suture to the suture anchor either (i) within the interior of a patient's body, or (ii) exterior to the patient's body. It will be appreciated that grappling the suture and slidably capturing the suture to the suture anchor within the interior of a patient's body can be highly advantageous, since it provides a fast, easy and reliable way to “pick up” the suture at an interior site. On the other hand, it will be appreciated that grappling the suture and slidably capturing the suture to the suture anchor exterior to the patient's body can also be highly advantageous, since it provides a fast, easy and reliable way to “pick up” the suture exterior to the patient's body.

In additional aspects of the present invention, bone hole 95 can be formed using a bone tunnel dilator (not shown) so as to compact the host bone and thereby form a more stable wall for the bone hole, whereby to enhance fixation.

Also, a groove (not shown) can be formed in the outer wall of elongated body 10, so as to provide a space to accommodate the suture: this can help reduce suture abrasion, particularly at the top end of the bone hole. However, it should be appreciated that seating suture 85 in a groove formed in exterior sidewall 30 of elongated body 10 may be reduce, or even eliminate entirely, binding of the suture between exterior sidewall 30 of elongated body 10 and the side wall of bone hole 95.

And suture anchor 5 can be formed with a thicker wall at the top end of the device, so as to assist fixation.

Also, a suture can have a repeating pattern of expansions, can be made especially coarse, or can carry a sleeve, etc. about a portion of its length so as to increase its effective diameter and thereby facilitate gripping by J-hook 75.

It is also possible to form elongated body with one or more fracture lines (or break points) along its length, so that diametrically-opposed side slots 40 of the elongated body may easily expand outwardly when actuator 15 is moved from its second, intermediate position to its third, retracted position. Furthermore, the side walls of elongated body 10 may include collapsible longitudinal channels for receiving a suture; these collapsible longitudinal channels help maintain the suture along elongated body 10 during insertion of suture anchor 5 into bone hole 95 and then, when actuator 15 is moved from its second intermediate portion to its third, retracted position, the collapsible longitudinal channels collapse so as to help secure suture 85 to elongated body 10 and thereby enhance binding of suture 85 to elongated body 10.

### Other Expandable Body Constructions, Including “Winged Body” Construction

With the suture anchor construction shown in FIGS. 1-16, the cross-sectional profile of elongated body 10 is enlarged (i.e., in order to bind the suture anchor in a bone hole) by splitting the elongated body along slots 40.

It is also possible to enlarge the cross-sectional profile of the suture anchor (i.e., in order to bind the suture anchor in a bone hole) by other means.

By way of example but not limitation, elongated body 10 may be formed without diametrically-opposed slots 40; in this form of the invention, elongated body 10 may be formed so that it is otherwise diametrically expandable (e.g., by fracturing open, by flexing open, etc.) when actuator 15 is longitudinally moved within elongated body 10. Thus, in this form of the invention, longitudinal movement of actuator 15 is still used to cam elongated body 10 diametrically outwardly, however, slots 40 are not provided to guide the manner of body expansion.

In one preferred form of the invention, the cross-sectional profile of elongated body 10 is enlarged (i.e., in order to bind the suture anchor in a bone hole) by selectively deploying wings outboard from the elongated body once the suture anchor has been disposed in a bone hole.

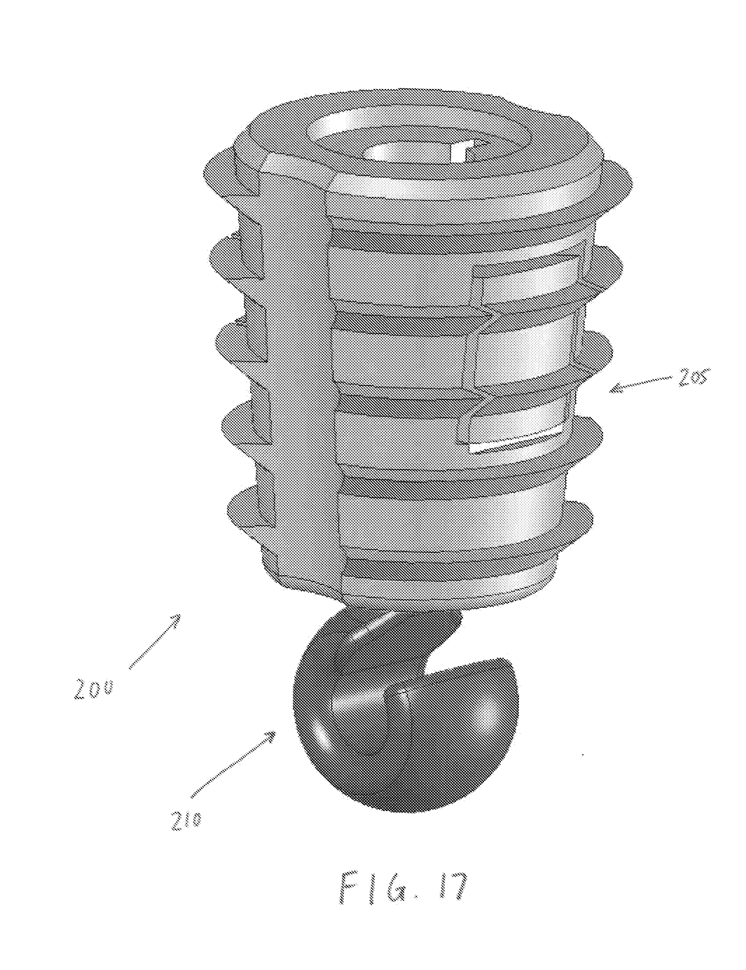

More particularly, and looking now at FIG. 17, there is shown a suture anchor 200 which generally comprises an elongated body 205 and an actuator 210.

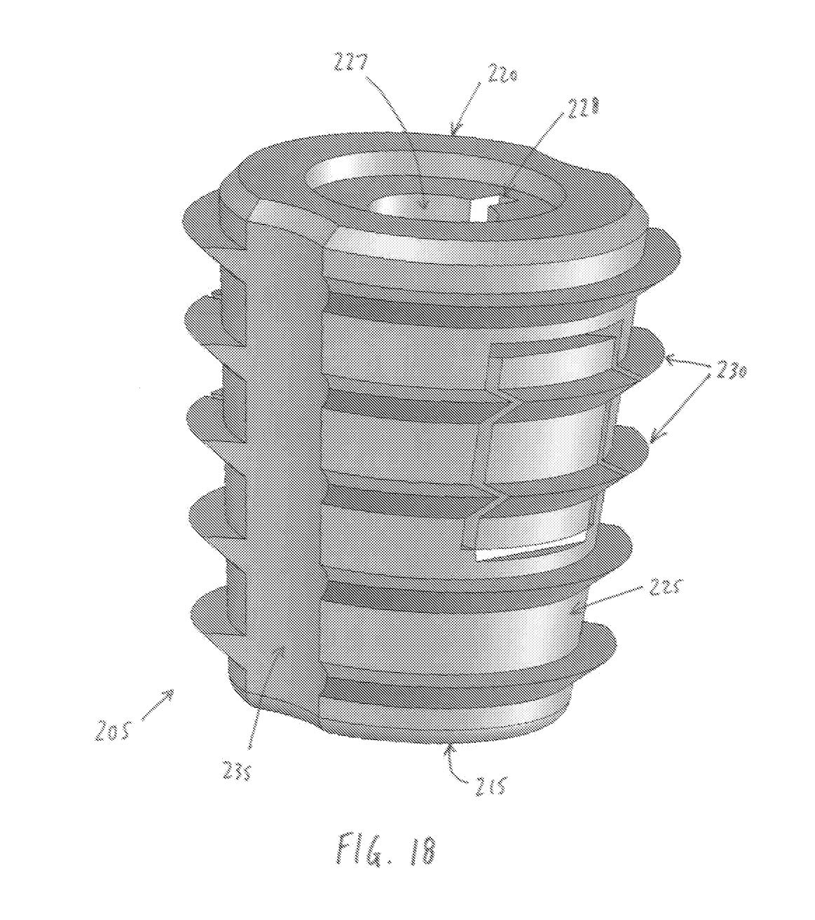

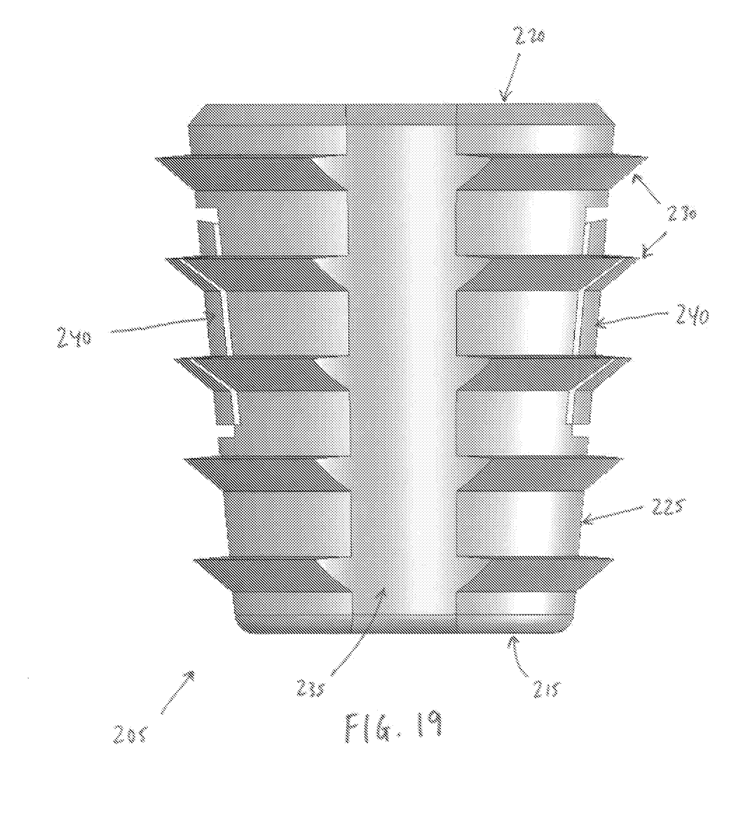

Elongated body 205 is shown in further detail in FIGS. 18 and 19, as well as in others of the figures. Elongated body 205 generally comprises a distal surface 215, a proximal surface 220 and an outer surface 225 connecting distal surface 215 to proximal surface 220. A bore 227 extends between distal surface 215 and proximal surface 220. Bore 227 includes a pair of diametrically-opposed longitudinal slots 228 extending therealong. Outer surface 225 preferably includes one or more ribs 230 extending circumferentially around the outer surface of elongated body 205, whereby to help engage the side wall of a bone hole, as will hereinafter be discussed. One or more longitudinally-extending grooves 235 may also be provided in outer surface 225. These longitudinally-extending grooves are preferably sized to receive one or more suture strands therein, as will also hereinafter be discussed. Elongated body 205 also comprises a pair of diametrically opposed wings 240. Wings 240 are hingedly connected to elongated body 205 so that they may selectively project outboard of elongated body 205, as will hereinafter be discussed in further detail.

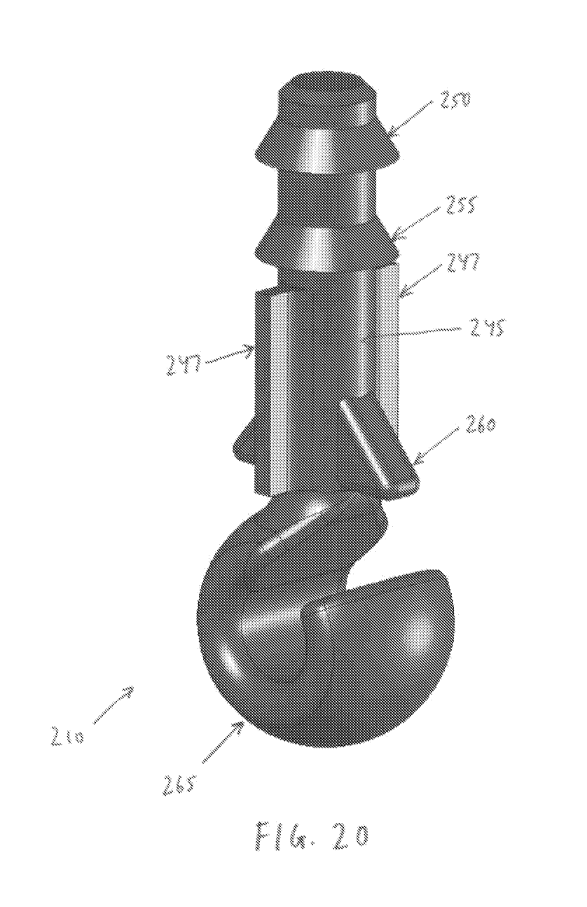

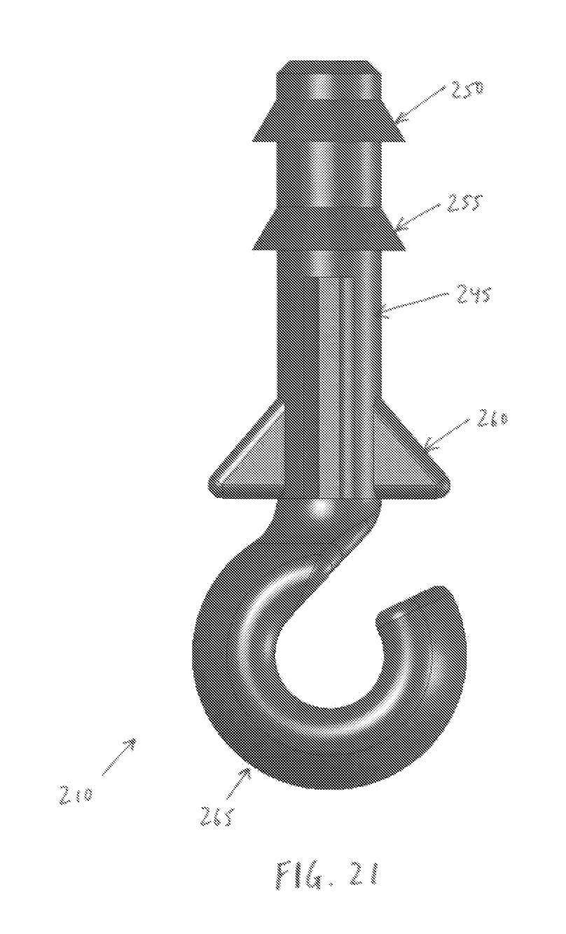

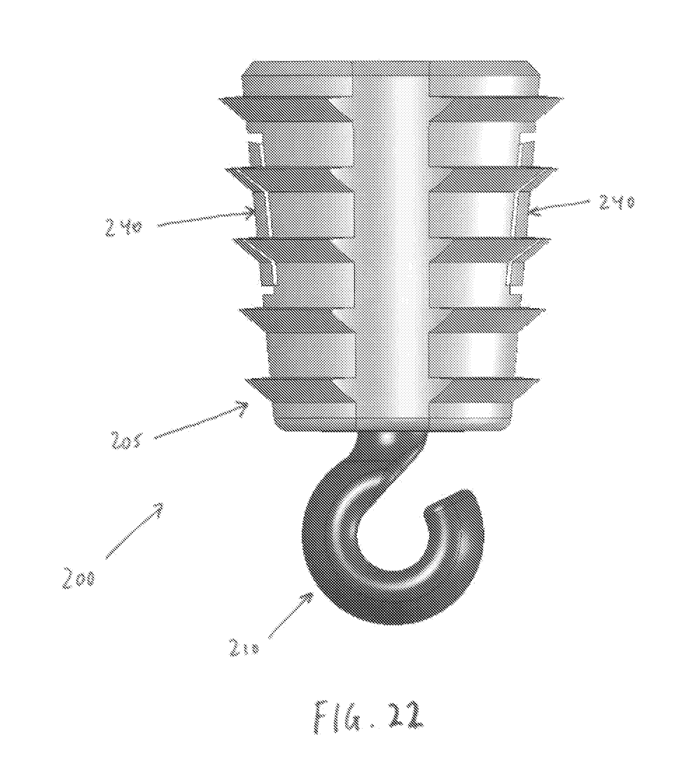

Actuator 210 is slidably disposed within bore 227 and diametrically-opposed longitudinal slots 228 of elongated body 205 as will hereinafter be discussed in further detail. Actuator 210 serves to engage one or more strands of suture, and to selectively deploy wings 240 of elongated body 205, as will hereinafter be discussed in further detail. Actuator 210 (FIGS. 20 and 21) generally comprises an elongated shaft 245 having a pair of longitudinally-extending tabs 247 extending therealong. A first tapered projection 250, a second tapered projection 255 and a third tapered projection 260 are disposed on shaft 245 and extend radially outward therefrom. Preferably first tapered projection 250 and second tapered projection 255 are frusto-conical in configuration, and third tapered projection 260 preferably comprises an inverted “arrowhead” configuration. A J-hook 265 is disposed at the distal end of shaft 245. As seen in FIGS. 21 and 22, J-hook 265 may comprise an arc extending in excess of 270 degrees, so as to provide a secure seat for engaging one or more strands of suture, as will hereinafter be discussed in further detail.

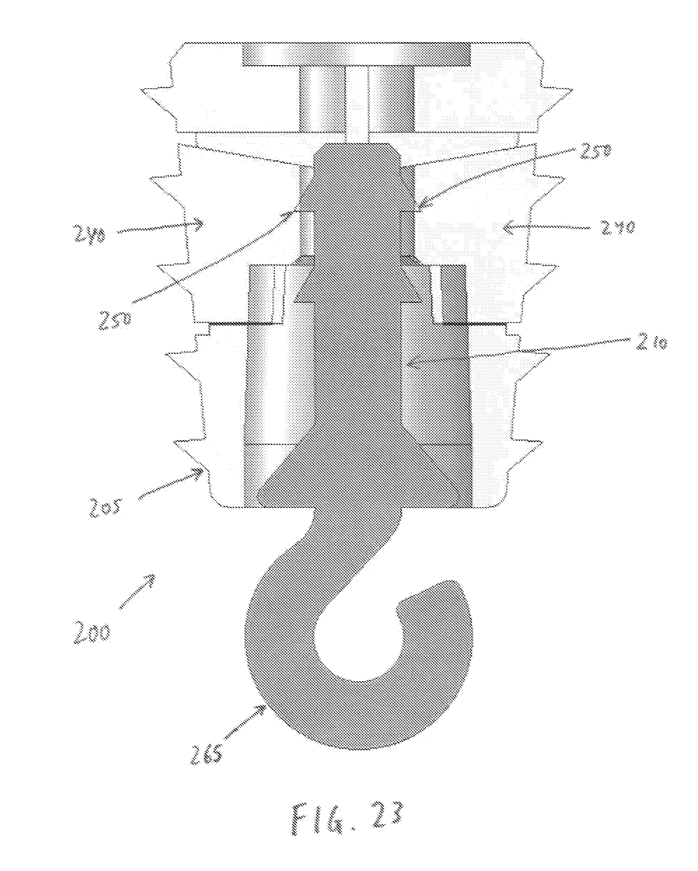

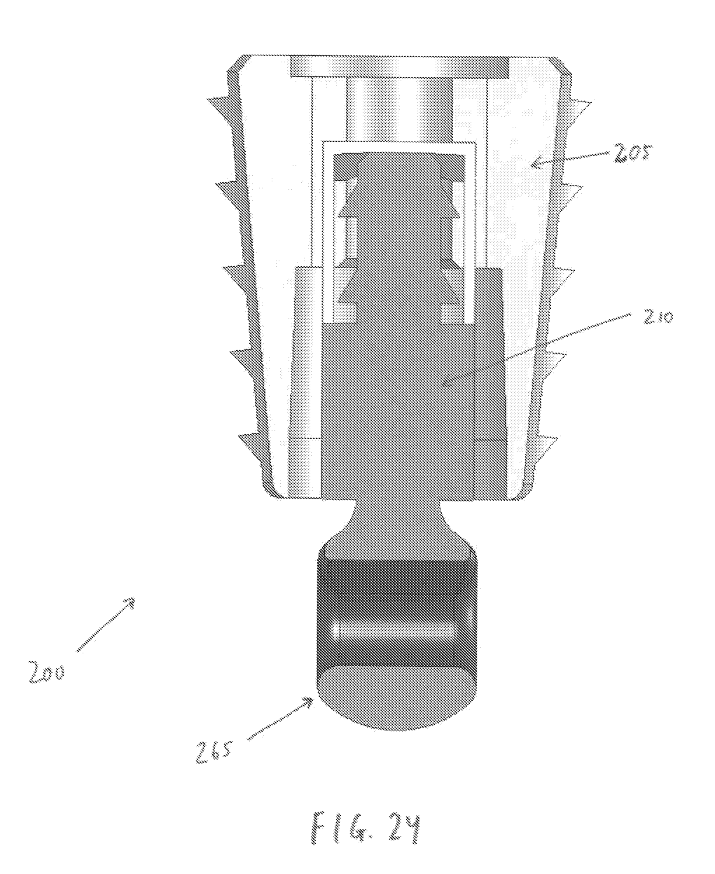

As seen in FIGS. 22-24, when actuator 210 is in its first, extended position, first tapered projection 250 of actuator 210 engages wings 240, with wings 240 being in their inboard position, and with J-hook 265 projecting a substantial distance out of the distal end of elongated body 205. In this first, extended position, J-hook 265 extends a sufficient distance beyond distal surface 215 such that J-hook 265 can be used to grapple one or more strands of suture.

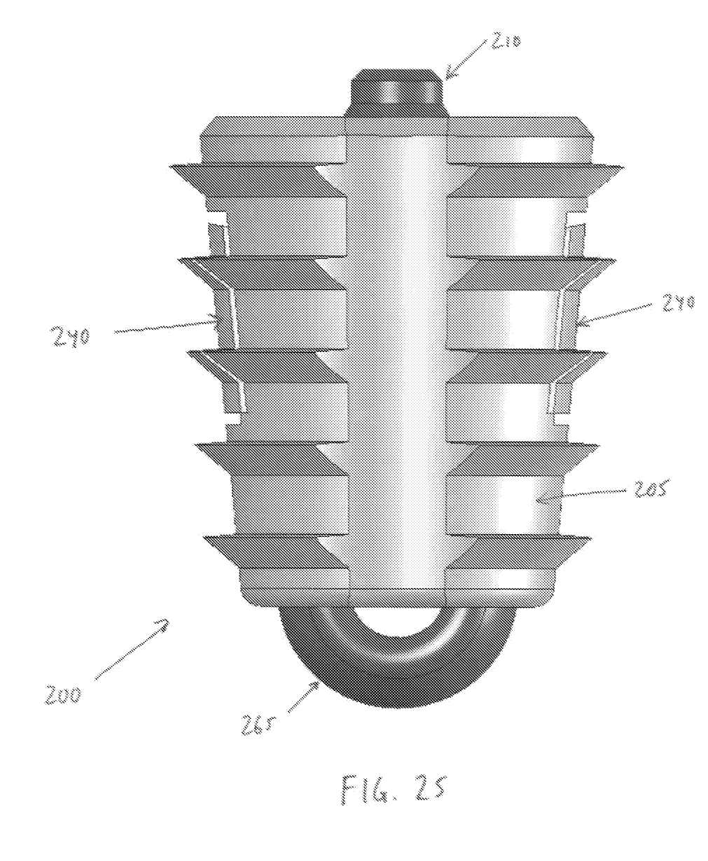

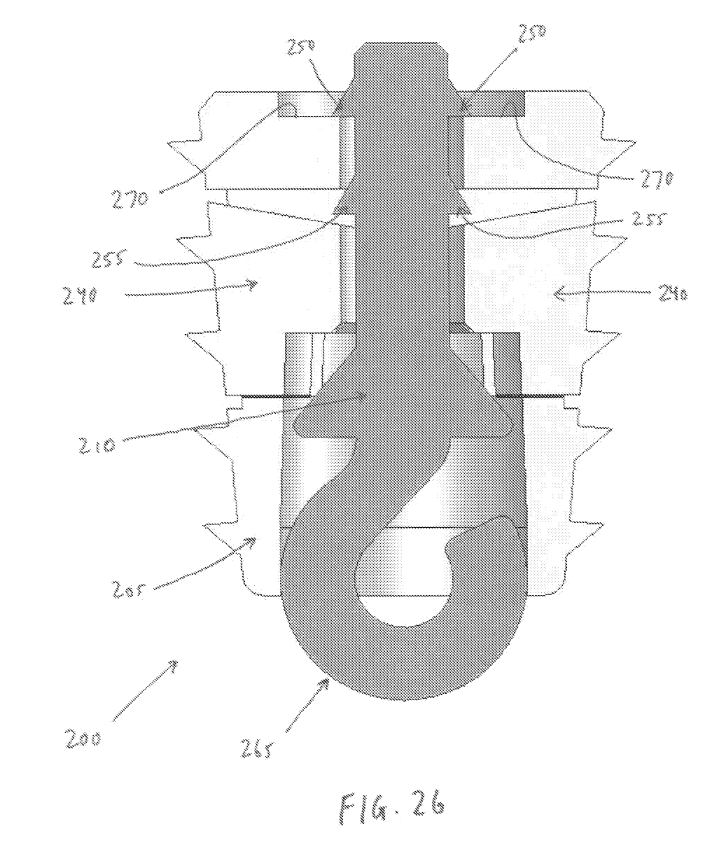

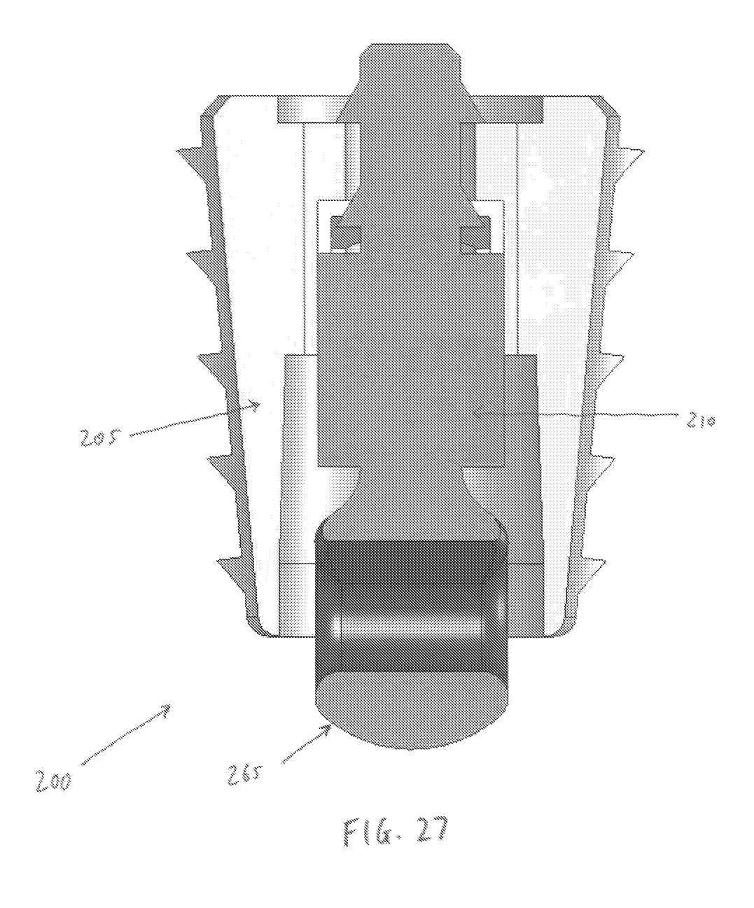

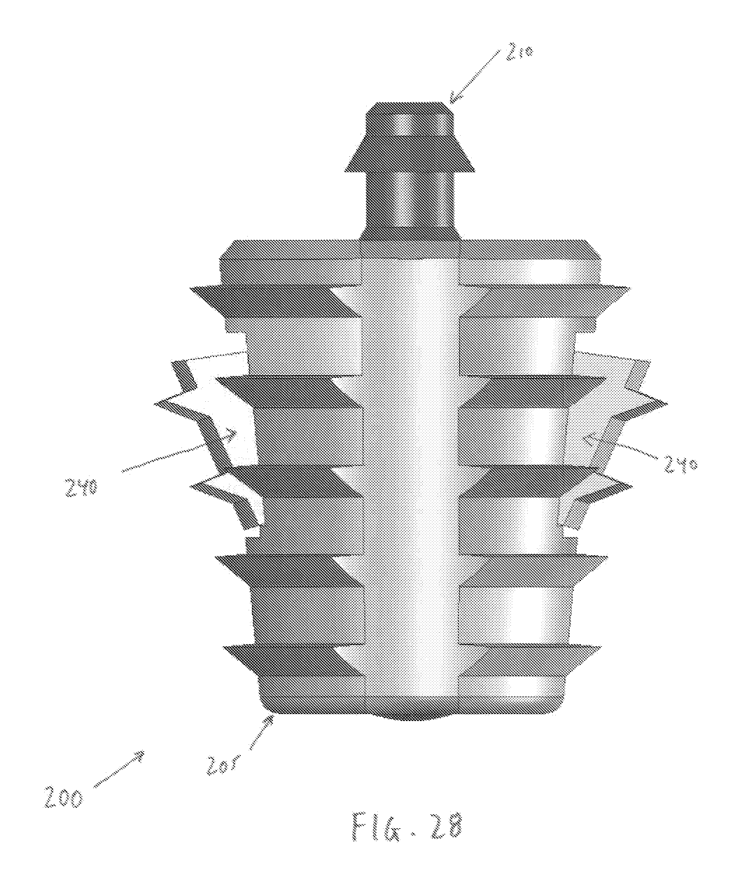

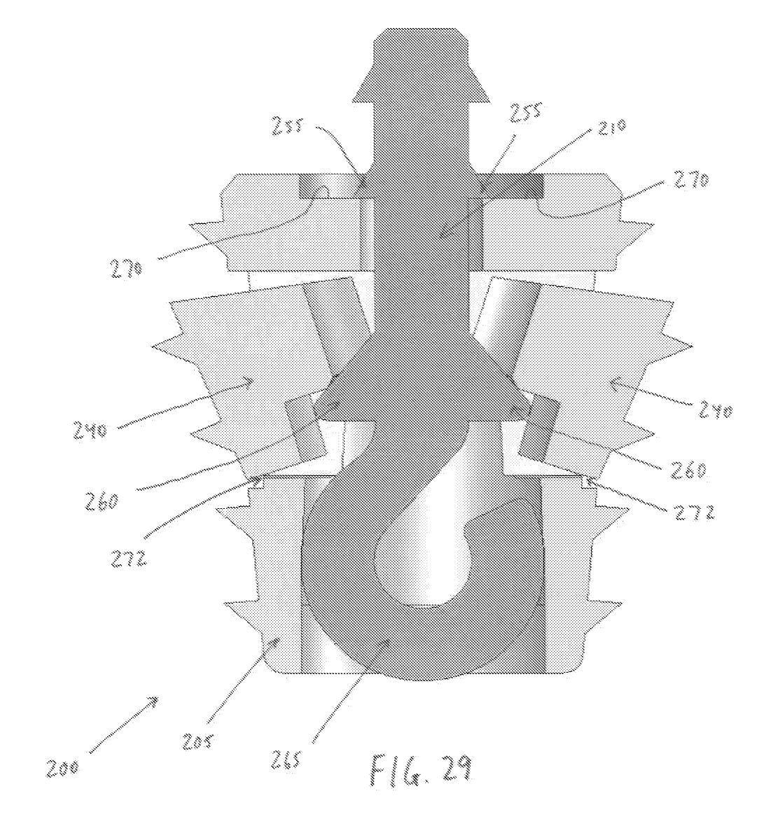

As seen in FIGS. 25-27, when actuator 210 is moved from its first, extended position to its second, intermediate position, first tapered projection 250 of actuator 210 will be seated in counterbore 270 of elongated body 205 and second tapered projection 255 will be disposed above wings 240, with wings 240 still being in their inboard position and with J-hook 265 projecting a small distance beyond distal surface 215 of elongated body 205. When actuator 210 is in its second, intermediate position, J-hook 265 is disposed relative to distal surface 215 such that one or more strands of suture may be slidably captured to the suture anchor.

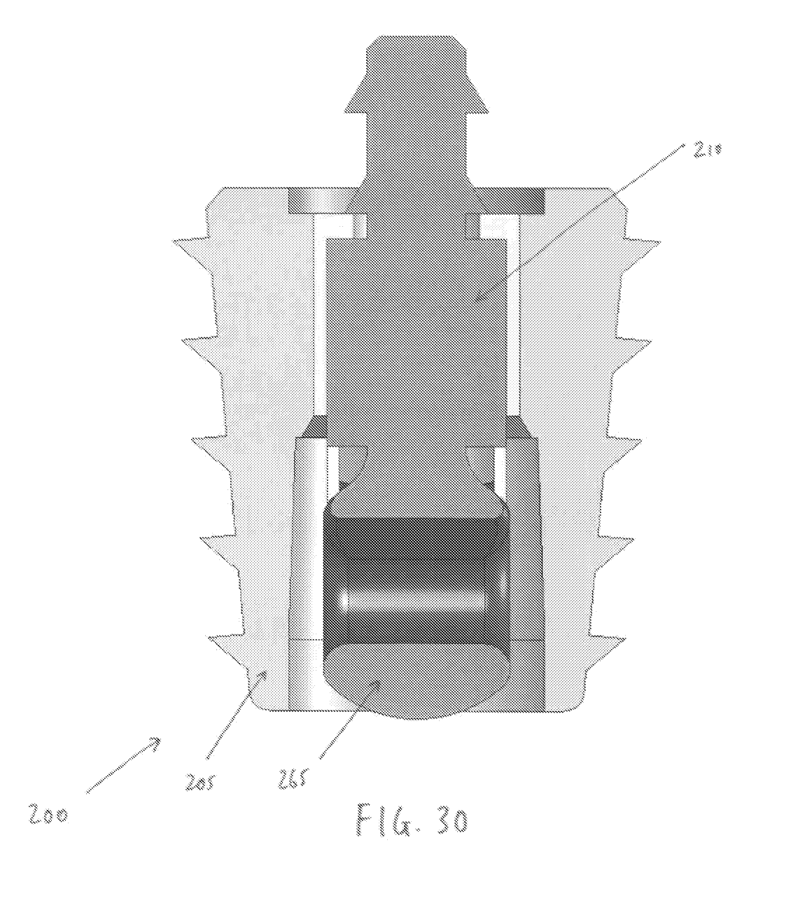

As seen in FIGS. 28-30, when actuator 210 moves from its second, intermediate position to its third, retracted position, third tapered projection 260 engages wings 240 and cams them outward, with wings 240 pivoting on their living hinges 272 so that wings 240 project aggressively outwardly from elongated body 205. When suture anchor 200 is in its third, retracted position, second tapered projection 255 is seated in counterbore 270 of elongated body 205, wings 240 are in their outboard position, and J-hook 265 is retracted up into elongated body 205. When J-hook 265 is retracted up into elongated body 205 in this manner, one or more suture strands disposed in J-hook 265 will be bound to elongated body 205.

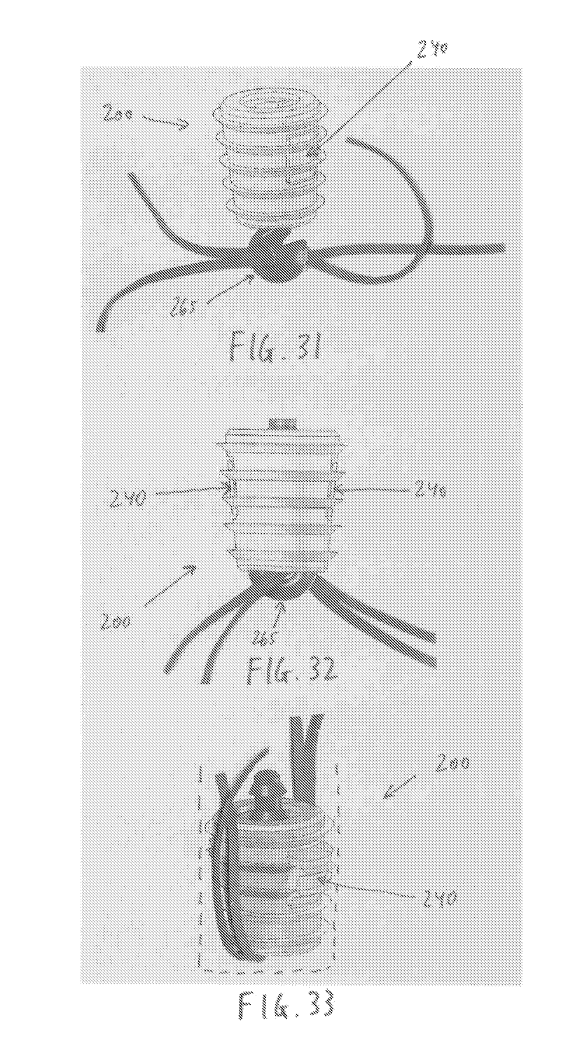

In use, and looking now at FIGS. 31-33, suture anchor 200 is first set so that its actuator 210 is in its first, extended position, and J-hook 265 is used to grapple one or more suture strands (FIG. 31); then suture anchor 200 is set so that its actuator 210 is in its second, intermediate position so as to slidably capture the suture to the suture anchor (FIG. 32), whereupon the suture anchor can be advanced into a bone hole, carrying the suture therewith; and finally the suture anchor is set by moving its actuator 210 into its third, retracted position (FIG. 33) so as to simultaneously (i) bind the suture strands to the suture anchor, and (ii) bind the suture anchor (via the expanded wings 240) in the bone hole.

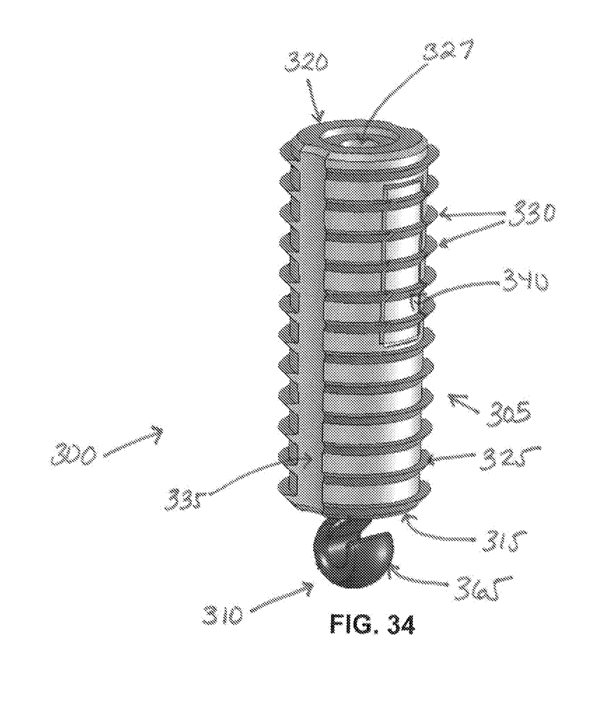

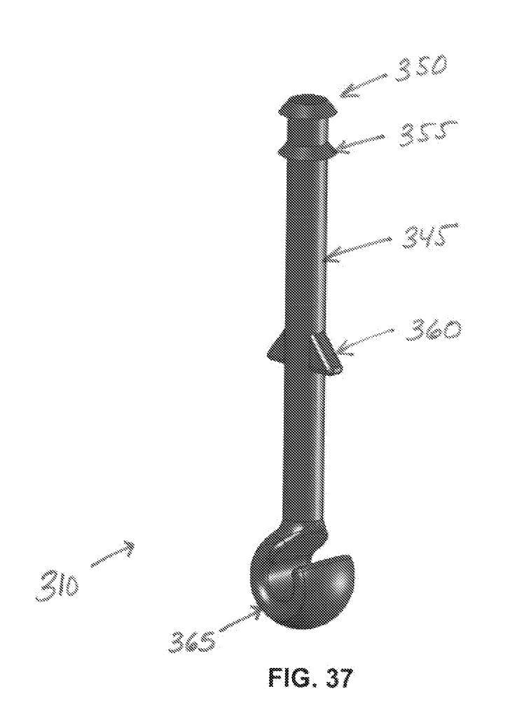

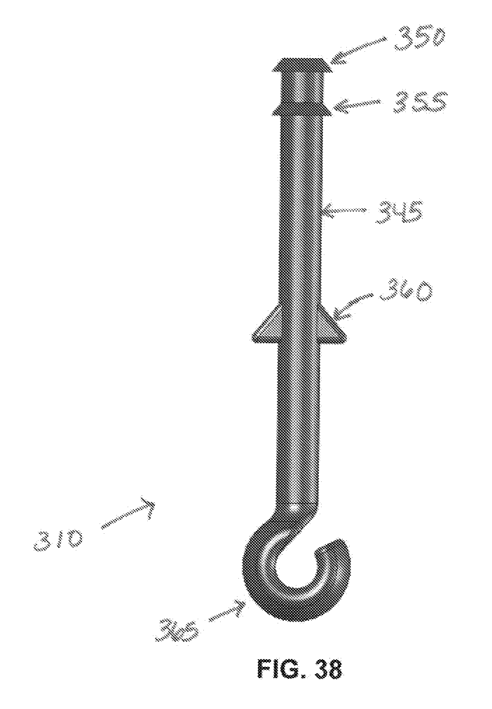

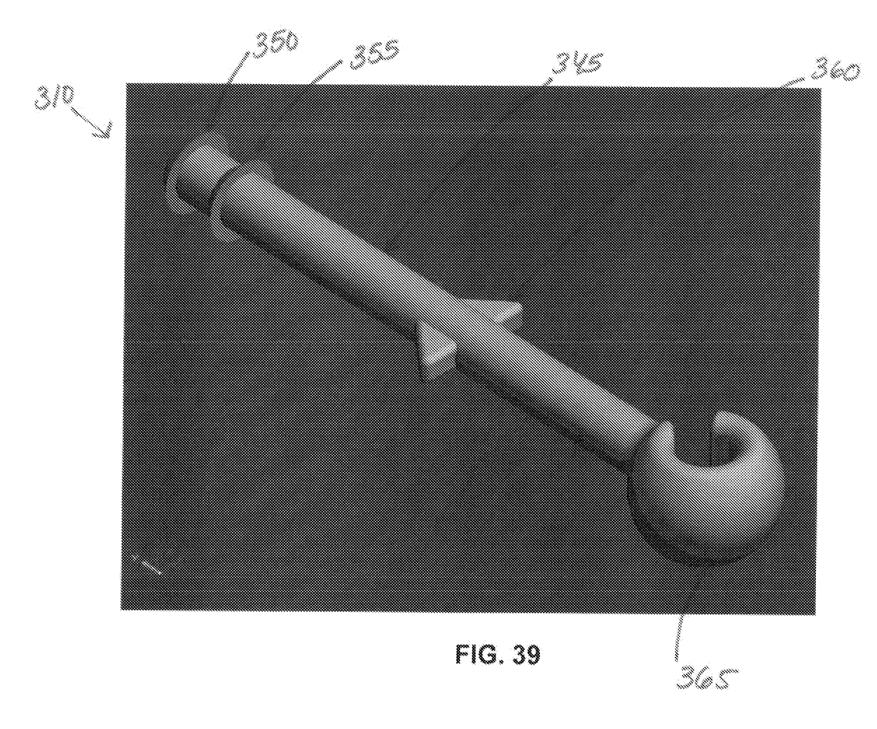

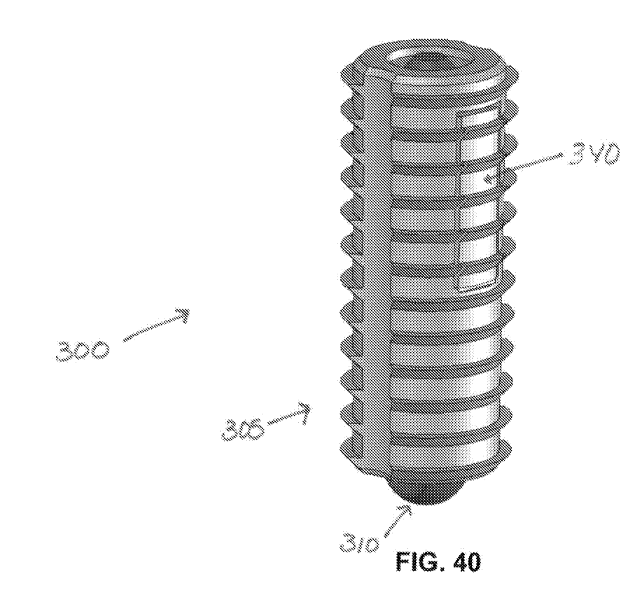

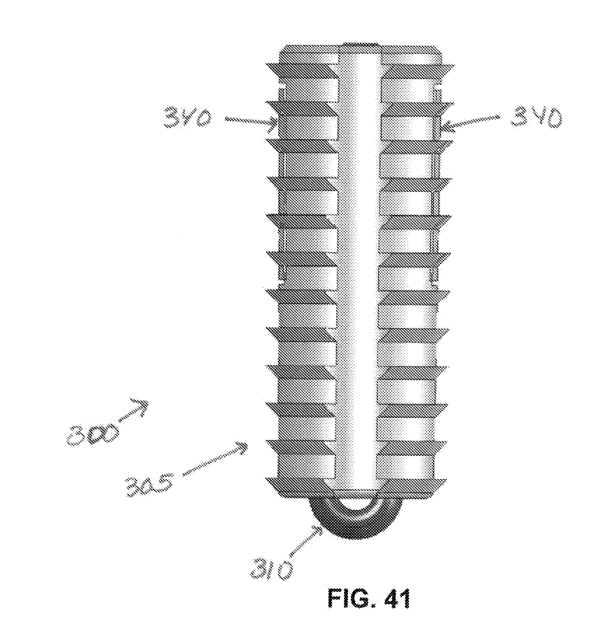

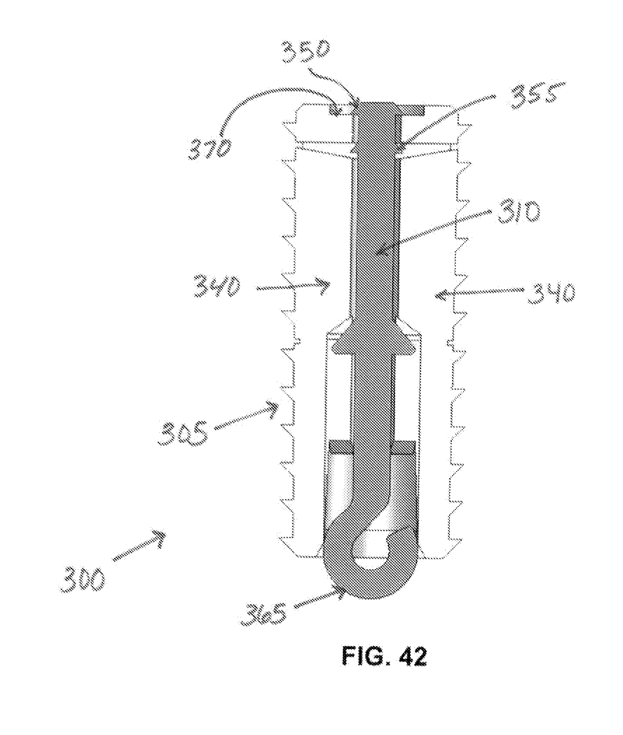

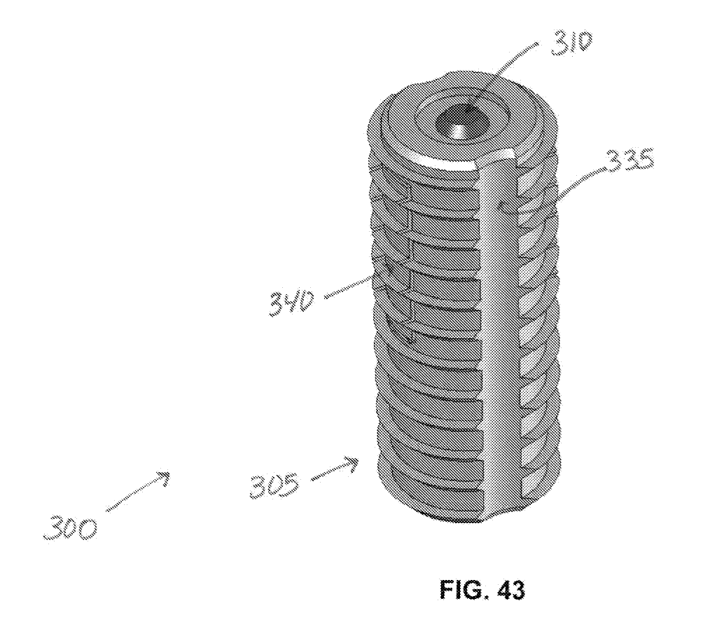

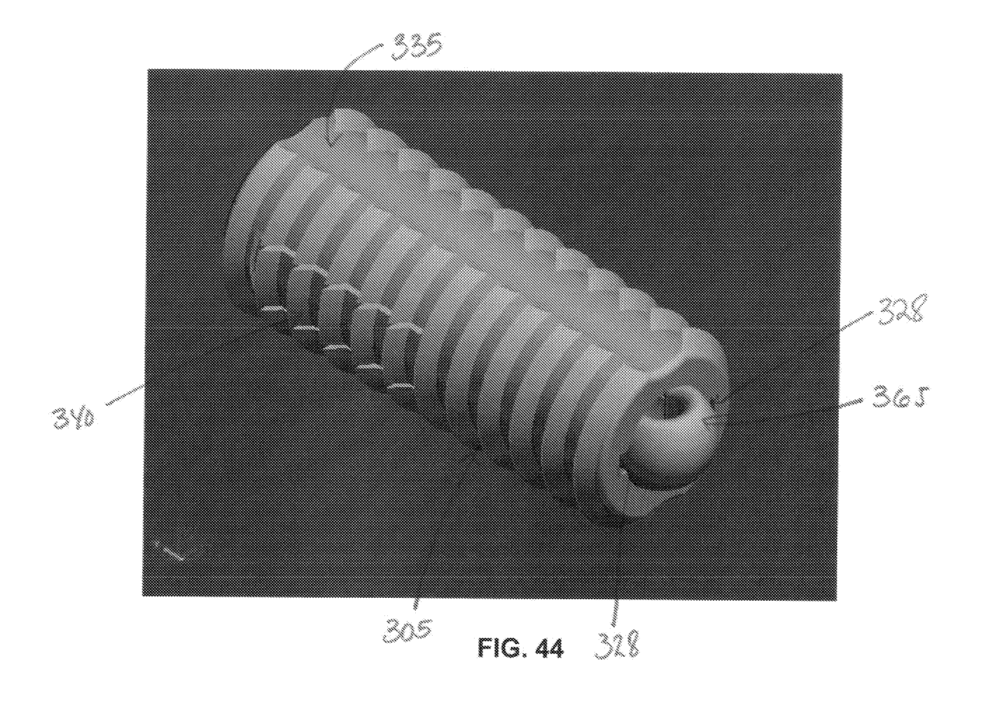

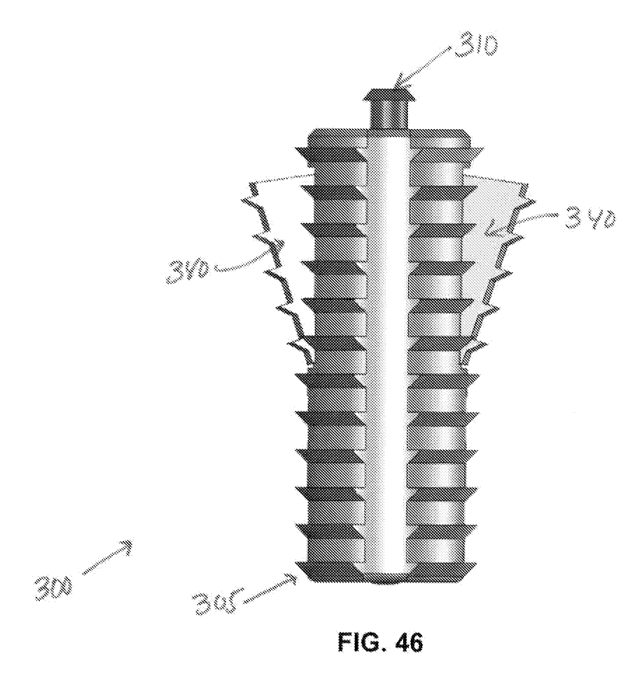

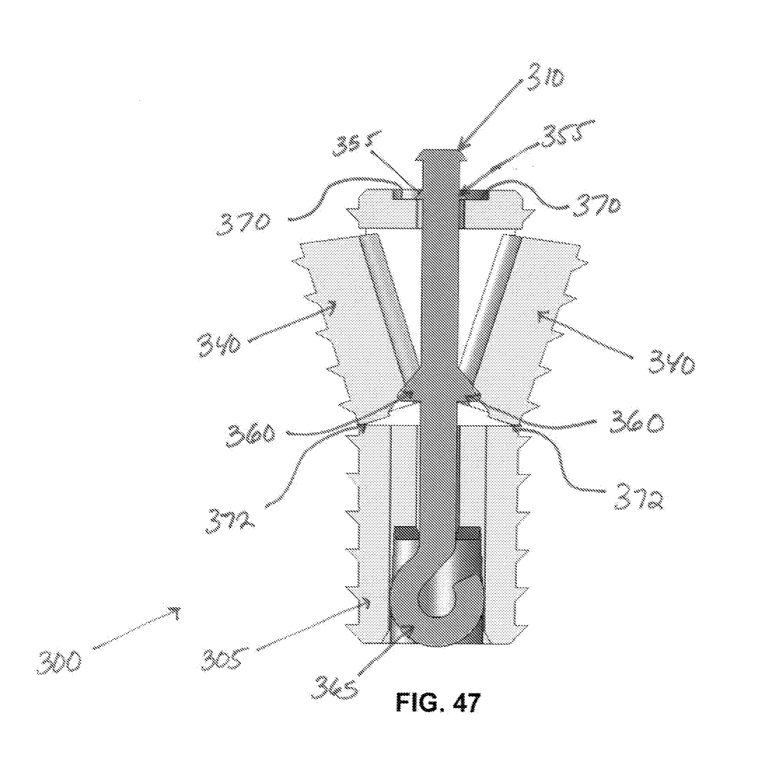

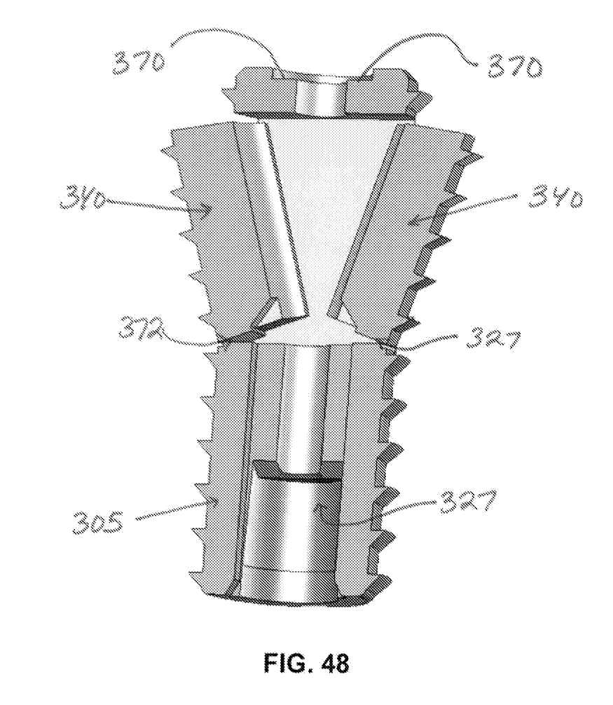

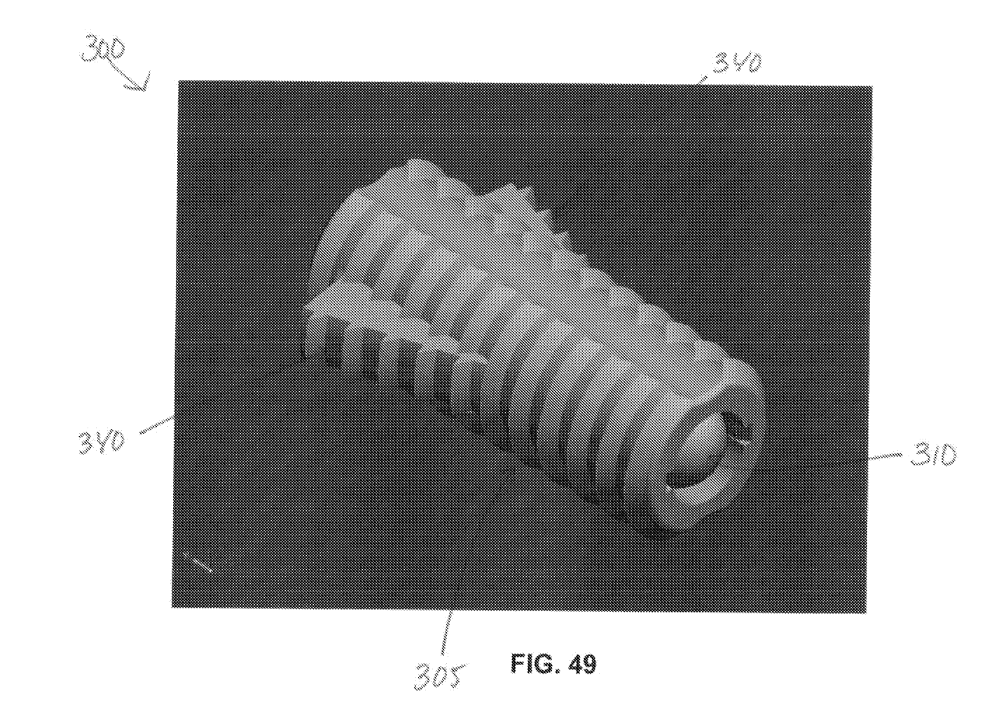

Looking next at FIGS. 34-49, there is shown another suture anchor 300 which also utilizes a “winged body” construction. Suture anchor 300 is similar to the suture anchor 200 described above, and generally comprises an elongated body 305 and an actuator 310.

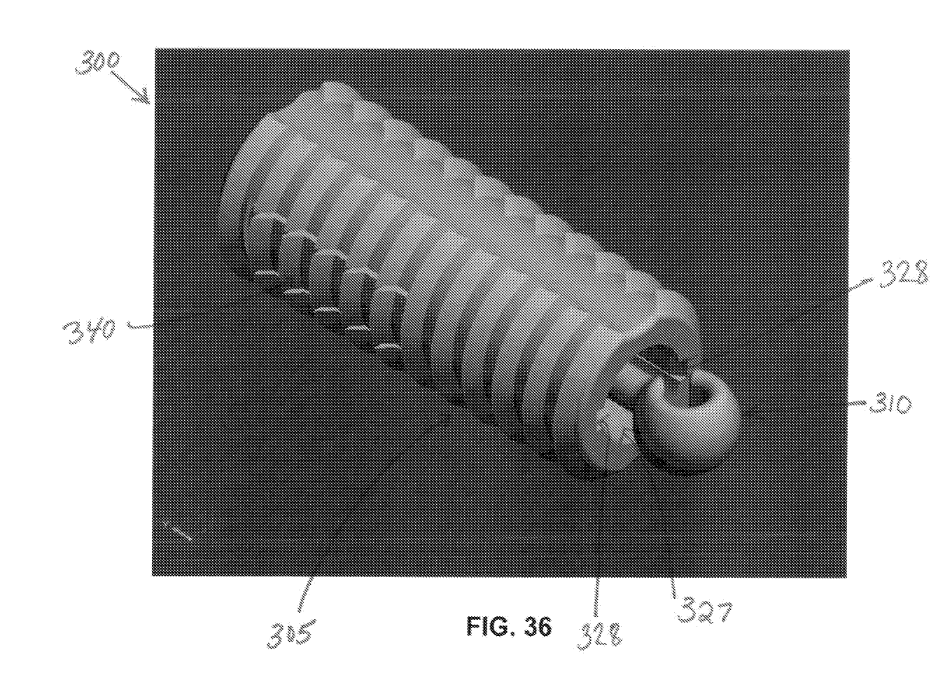

Elongated body 305 generally comprises a distal surface 315, a proximal surface 320 and an outer surface 325 connecting distal surface 315 to proximal surface 320. A bore 327 extends between distal surface 315 and proximal surface 320. Bore 327 includes a pair of diametrically-opposed longitudinal slots 328 (FIG. 36) extending along the distal portion of bore 327. Outer surface 325 preferably includes one or more ribs 330 extending circumferentially around the outer surface of elongated body 305, whereby to help engage the side wall of a bone hole, as will hereinafter be discussed. One or more longitudinally-extending grooves 335 may also be provided in outer surface 325. These longitudinally-extending grooves are preferably sized to receive one or more suture strands therein, as will also hereinafter be discussed. Elongated body 305 also comprises a pair of diametrically opposed wings 340. Wings 340 are hingedly connected to elongated body 305 so that they may selectively project outboard of elongated body 305, as will hereinafter be discussed in further detail.

Actuator 310 is slidably disposed within bore 327 and diametrically-opposed longitudinal slots 328 of elongated body 305 as will hereinafter be discussed in further detail. Actuator 310 serves to engage one or more strands of suture, and to selectively deploy wings 340 of elongated body 305, as will hereinafter be discussed in further detail. Actuator 310 generally comprises an elongated shaft 345. A first tapered projection 350, a second tapered projection 355 and a third tapered projection 360 are disposed on shaft 345 and extend radially outward therefrom. Preferably first tapered projection 350 and second tapered projection 355 are frusto-conical in configuration, and third tapered projection 360 preferably comprises an inverted “arrowhead” configuration. A J-hook 365 is disposed at the distal end of shaft 345. J-hook 365 may comprise an arc extending in excess of 270 degrees, so as to provide a secure seat for engaging one or more strands of suture, as will hereinafter be discussed in further detail.

When actuator 210 is in its first, extended position (FIGS. 34-36), first tapered projection 350 of actuator 310 engages wings 340, with wings 340 being in their inboard position, and with J-hook 365 projecting a substantial distance out of the distal end of elongated body 305. In this first, extended position, J-hook 365 extends a sufficient distance beyond distal surface 315 such that J-hook 365 can be used to grapple one or more strands of suture.

When actuator 310 is moved from its first, extended position to its second, intermediate position (FIGS. 40-44), first tapered projection 350 of actuator 310 will be seated in counterbore 370 of elongated body 305 and second tapered projection 355 will be disposed above wings 340, with wings 340 still being in their inboard position and with J-hook 365 projecting a small distance beyond distal surface 315 of elongated body 305. When actuator 310 is in its second, intermediate position, J-hook 365 is disposed relative to distal surface 315 such that one or more strands of suture may be slidably captured to the suture anchor.

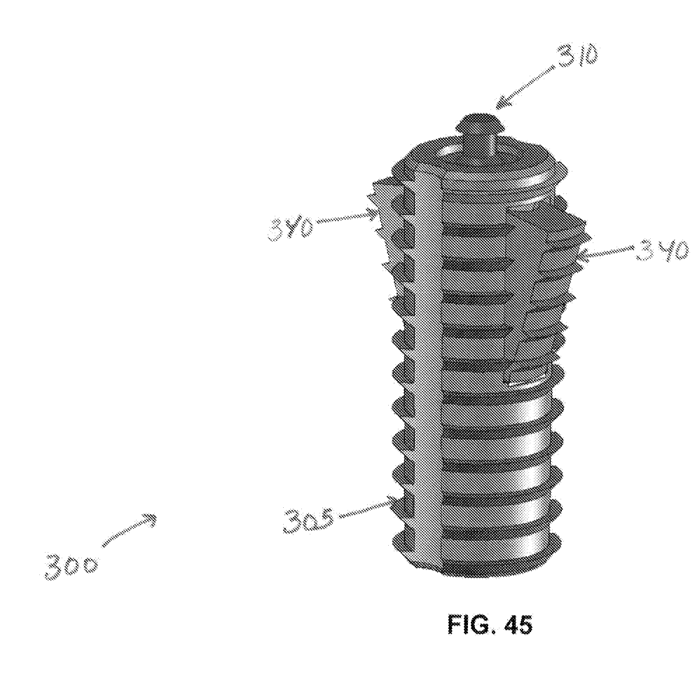

When actuator 310 moves from its second, intermediate position to its third, retracted position (FIGS. 45-49, third tapered projection 360 engages wings 340 and cams them outward, with wings 340 pivoting on their living hinges 372 so that wings 340 project aggressively outwardly from elongated body 305. When suture anchor 300 is in its third, retracted position, second tapered projection 355 is seated in counterbore 370 of elongated body 305, wings 340 are in their outboard position, and J-hook 365 is retracted up into elongated body 305. When J-hook 365 is retracted up into elongated body 305 in this manner, one or more suture strands disposed in J-hook 365 will be bound to elongated body 305.

Thus it will be seen that, when suture anchor 300 is fully deployed within a bone hole, three different types of fastening will occur: (i) elongated body 305 will be secured to the side wall of the bone hole due to the deployment of wings 340, (ii) suture 85 will be bound to elongated body 305 due to the retraction of actuator 310 into the interior of elongated body 305, and (iii) suture 85 may or may not be pinched between elongated body 305 and the side wall of the bone hole, depending on the diameter of the suture and the depth of the longitudinally-extending grooves 335.

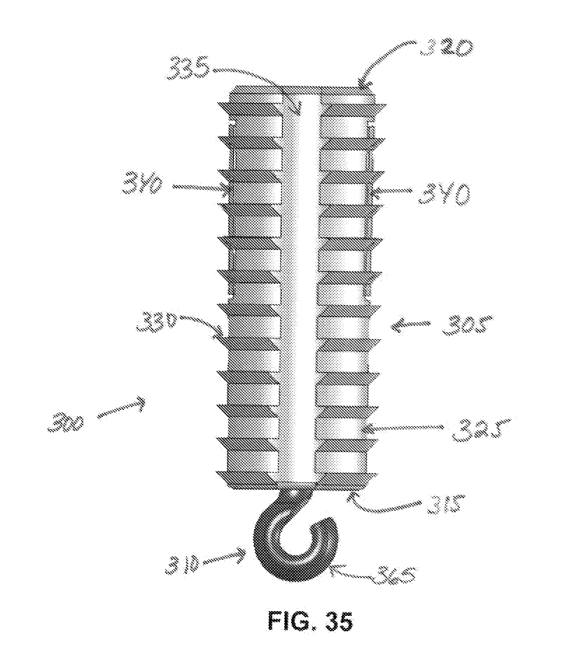

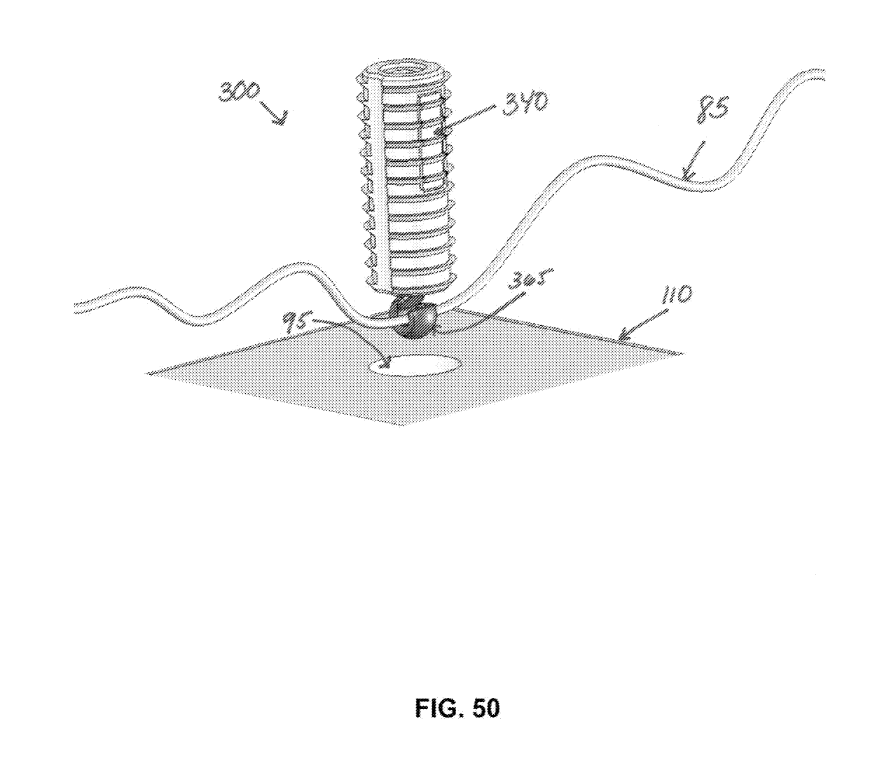

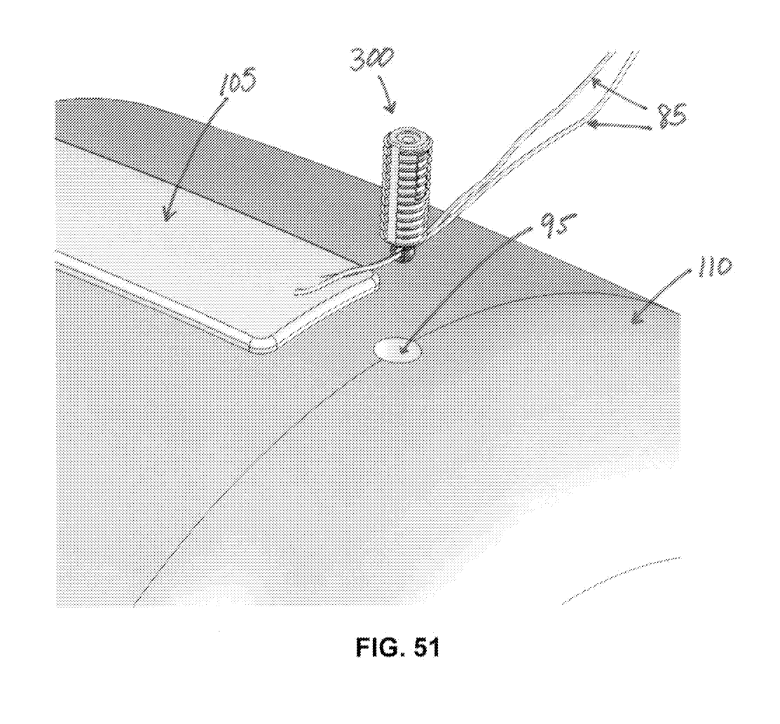

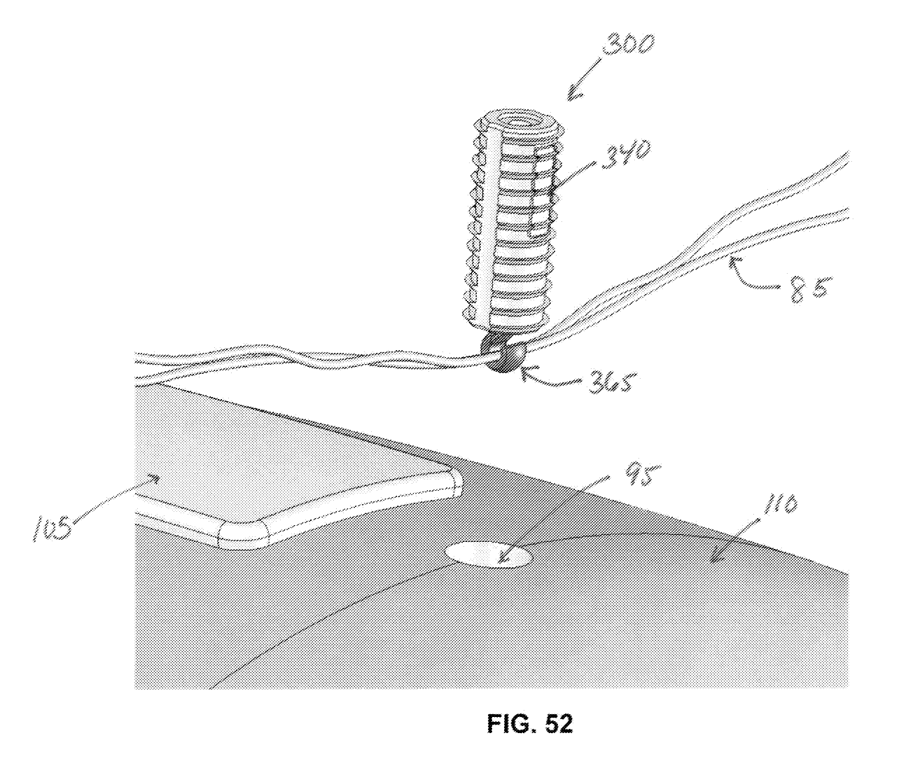

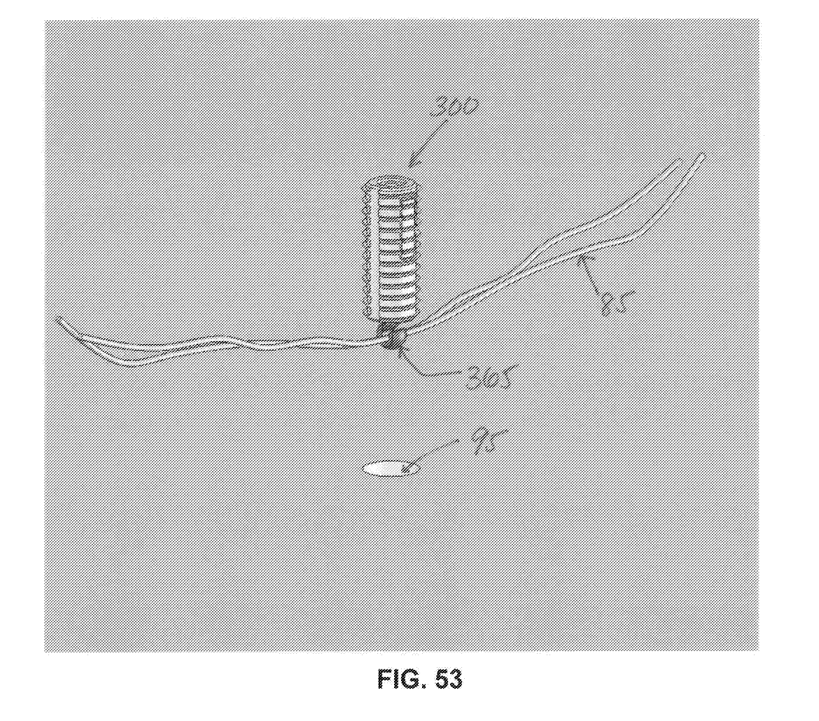

In use, and looking first at FIGS. 50-53, bone hole 95 is first formed in a host bone 110, suture anchor 300 has its actuator 310 positioned in its first, extended position (i.e., the position shown in FIGS. 34 and 35), suture 85 is passed through an object 105 (e.g., a rotator cuff) which is to be attached to bone 110, the two free ends of suture 85 are placed parallel to one another and tensioned (e.g., so as to pull object 105 into position relative to bone 110) and then, while tension is maintained on suture 85, J-hook 365 is used to hook (e.g., grapple) suture 85.

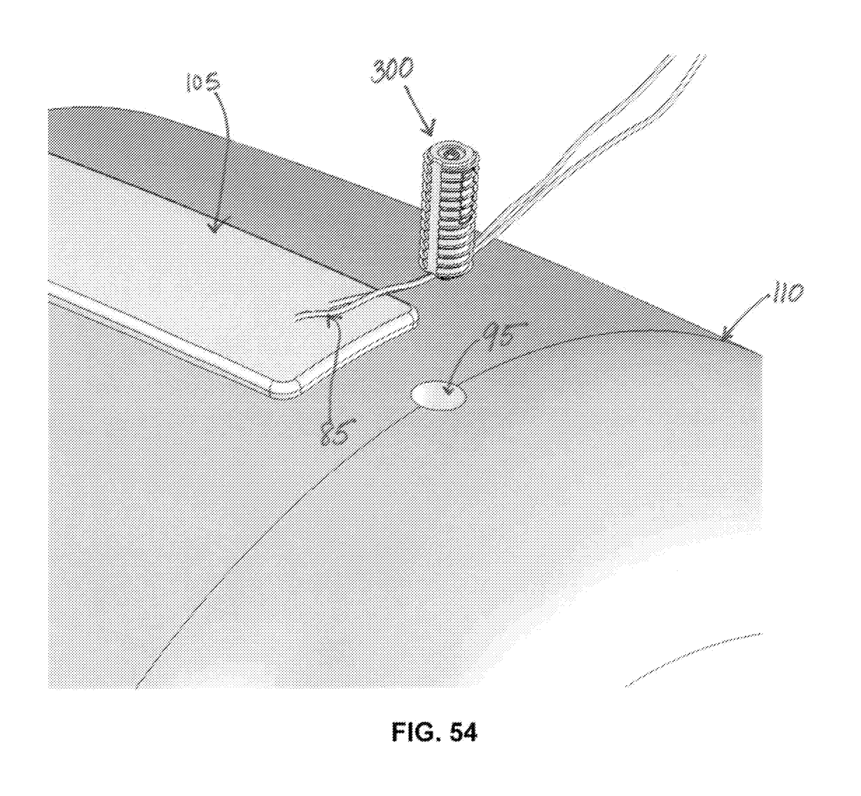

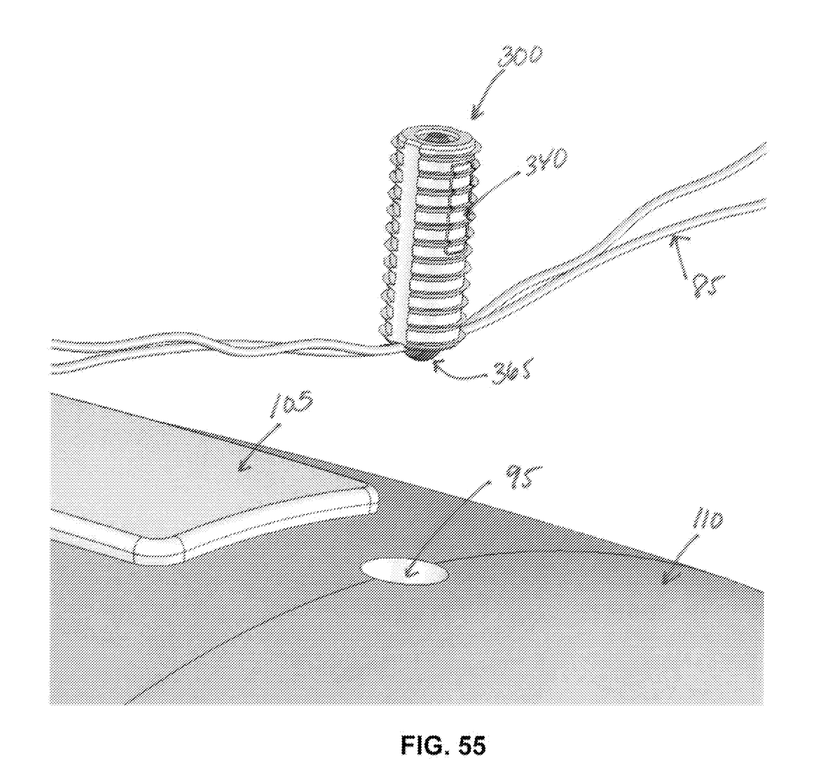

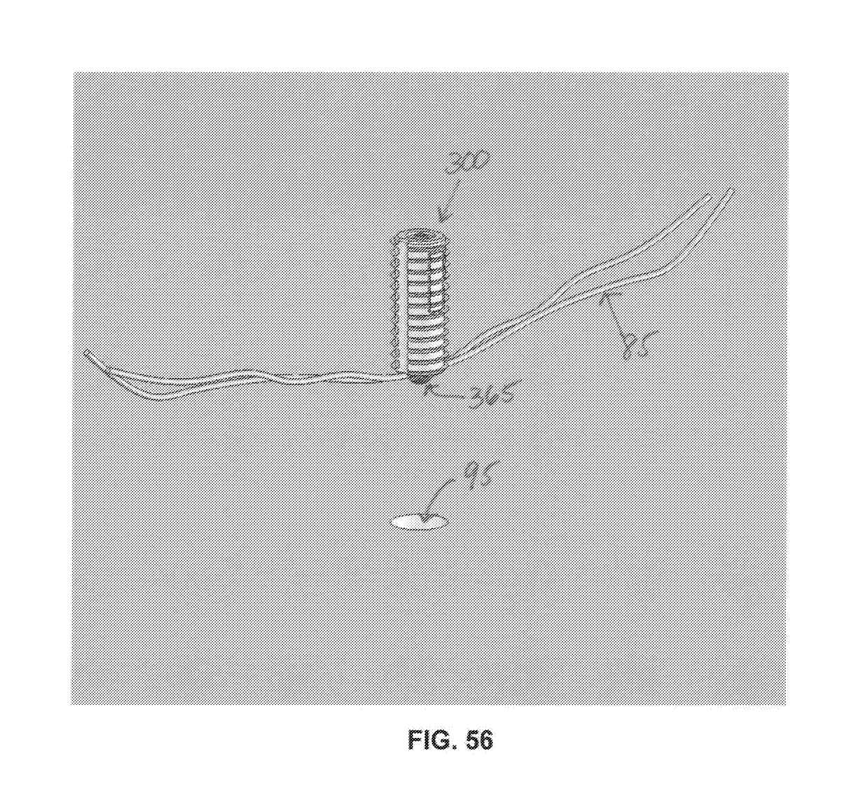

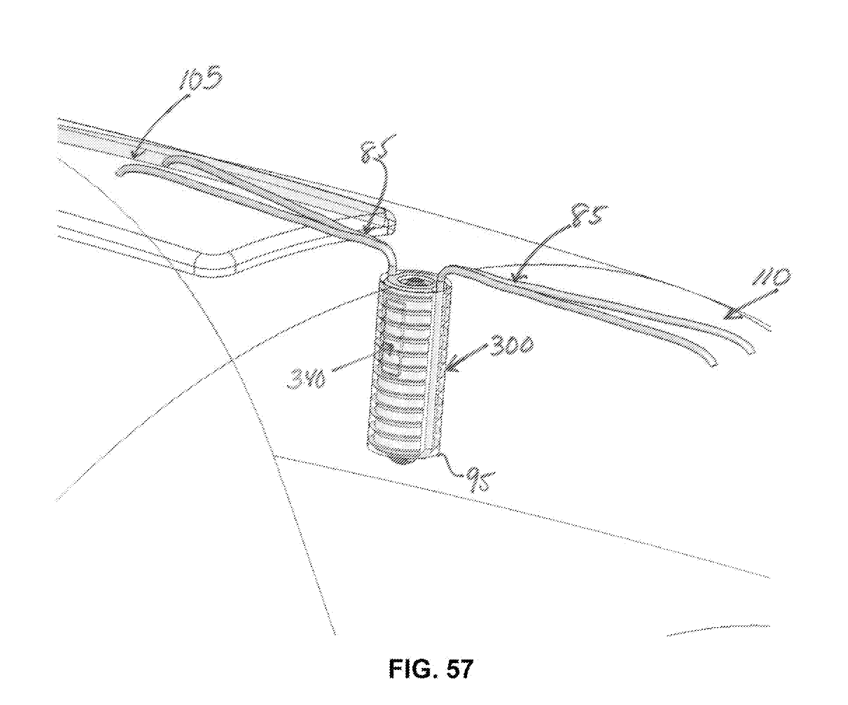

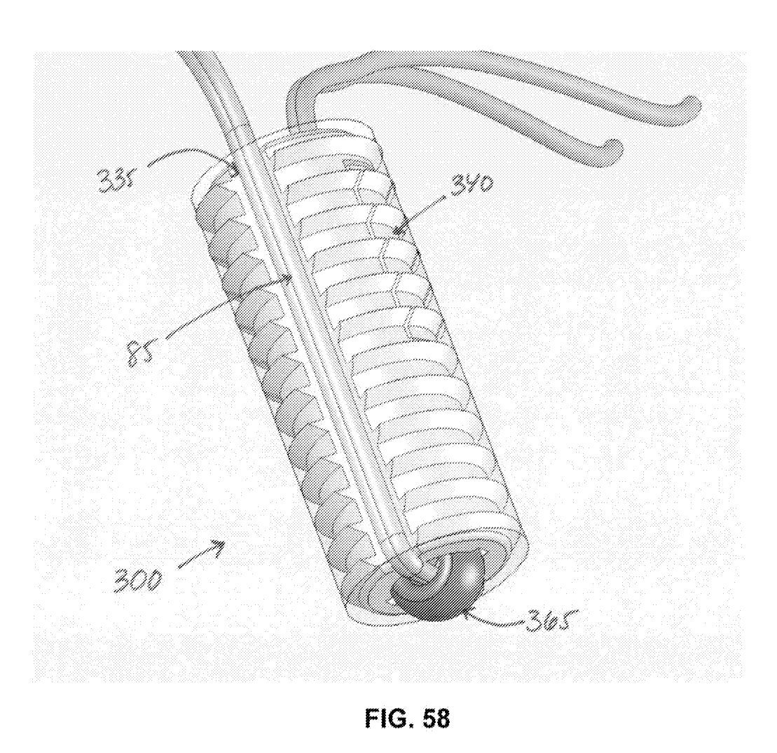

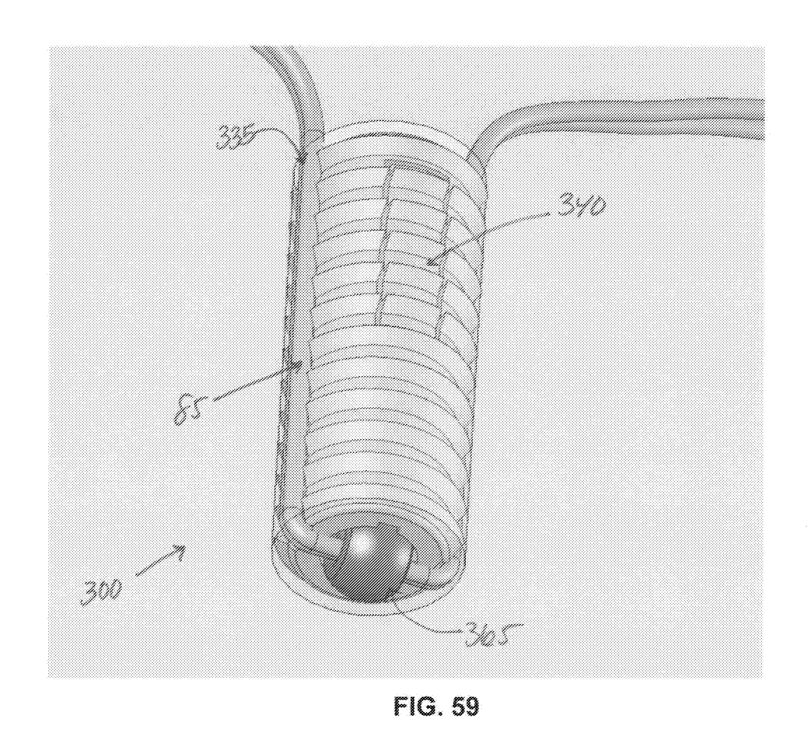

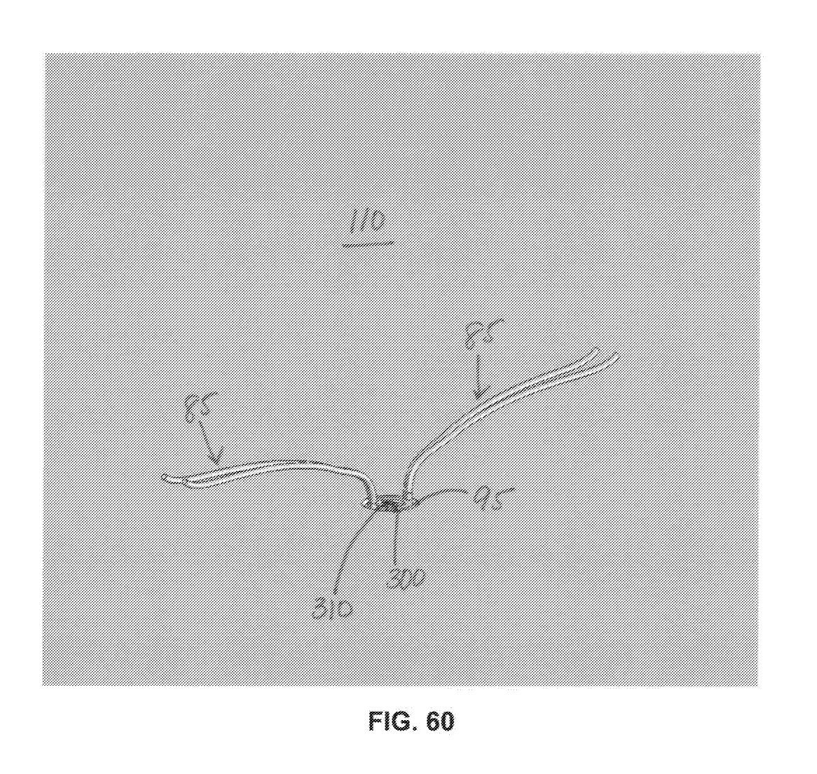

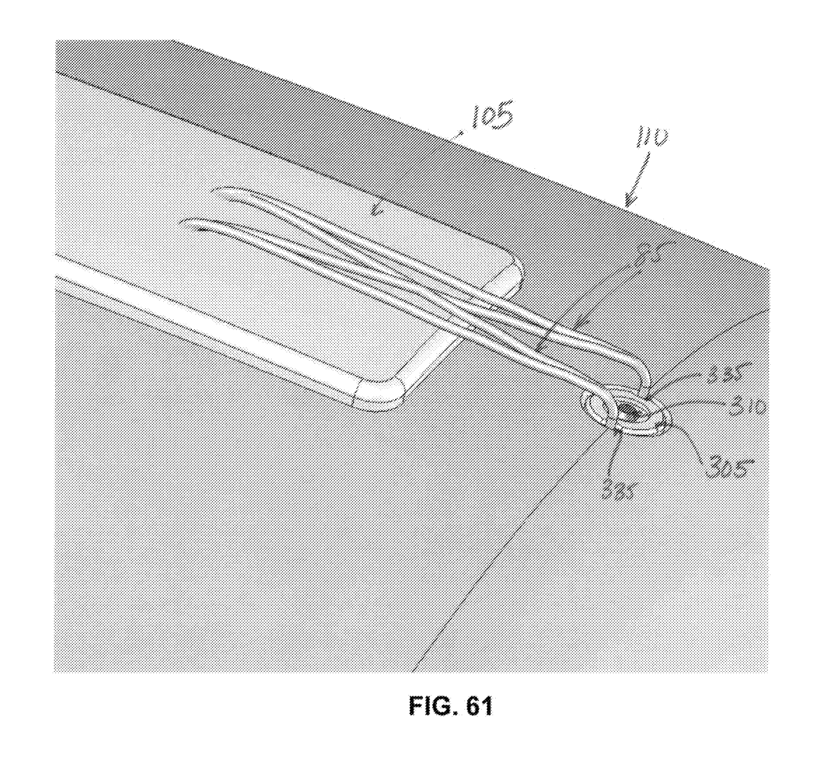

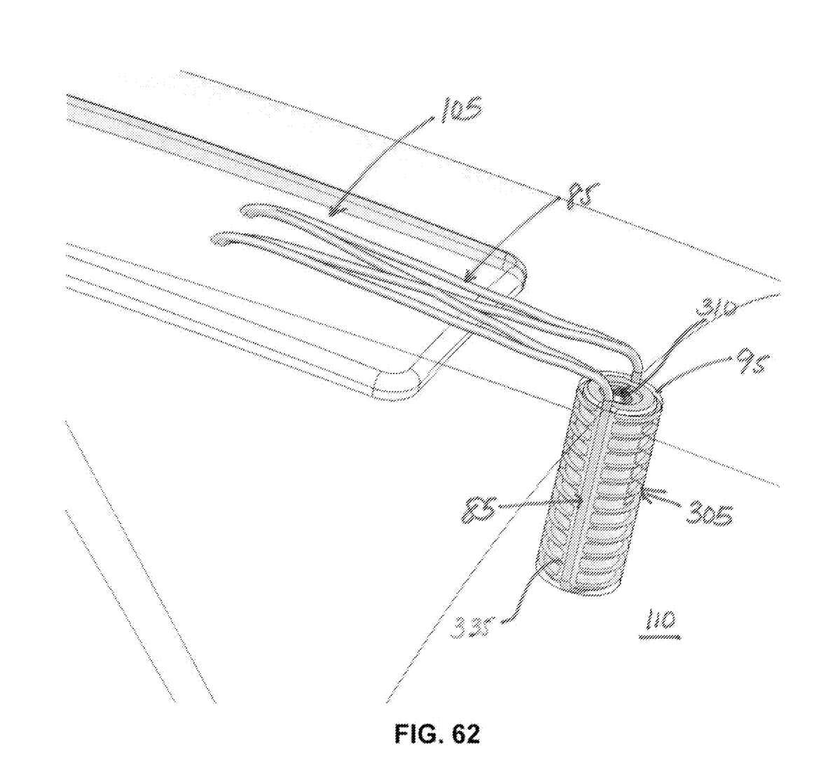

Next, and looking now at FIGS. 54-56, actuator 310 of suture anchor 300 is moved from its first, extended position to its second, intermediate position (i.e., the position shown in FIGS. 40-44) so that suture 85 is slidably captured between J-hook 365 and distal end surface 315 of elongated body 305. Then elongated body 305 is inserted into bone hole 95, with suture 85 extending within longitudinally-extending grooves 335 of elongated body 305 (FIGS. 57-62). In this respect it will be appreciated that suture anchor 300 will slip easily into bone hole 95, since wings 340 have not yet been radially projected out of the anchor body.

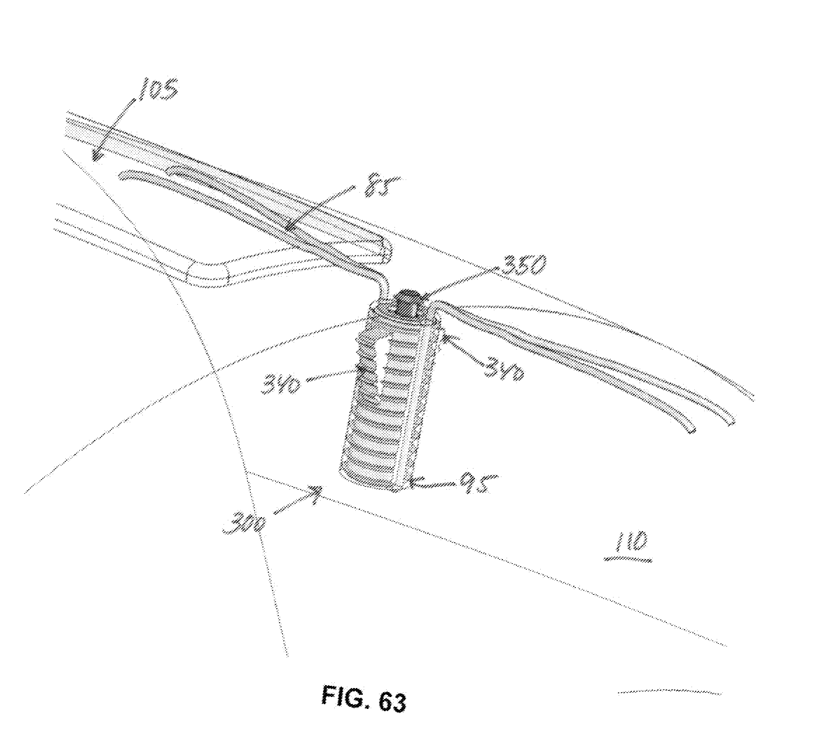

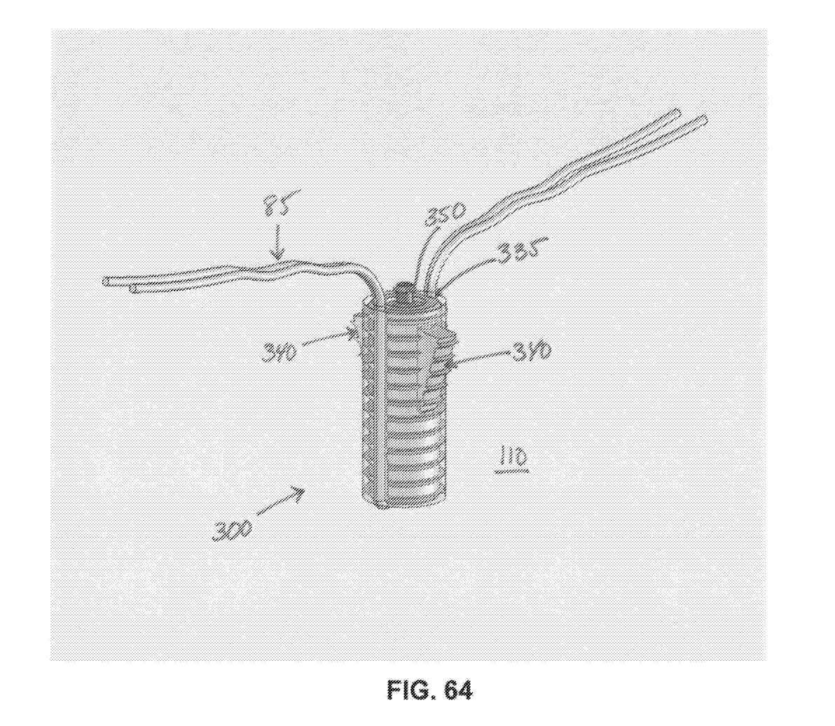

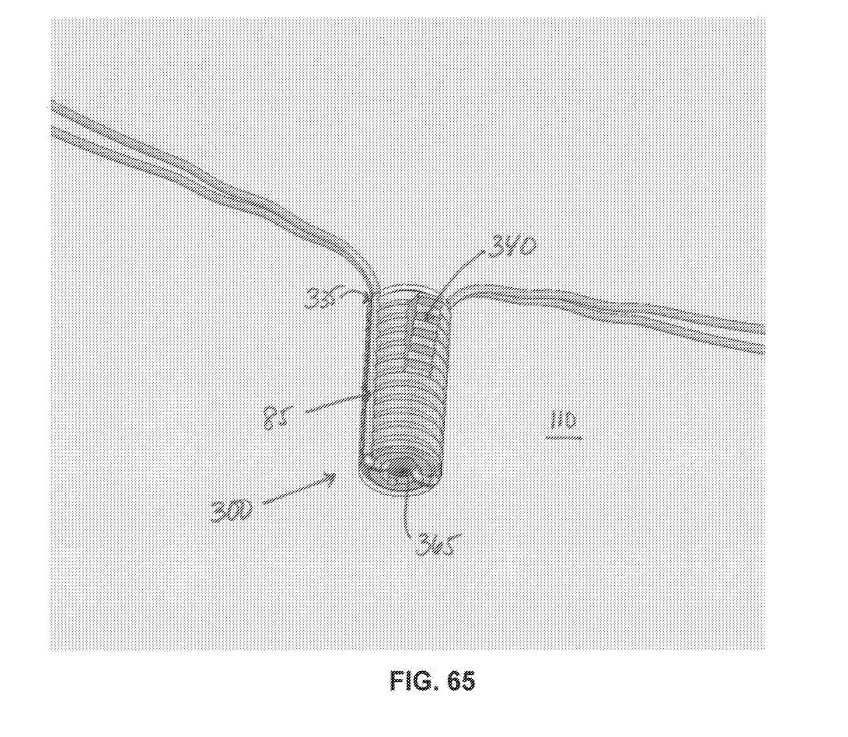

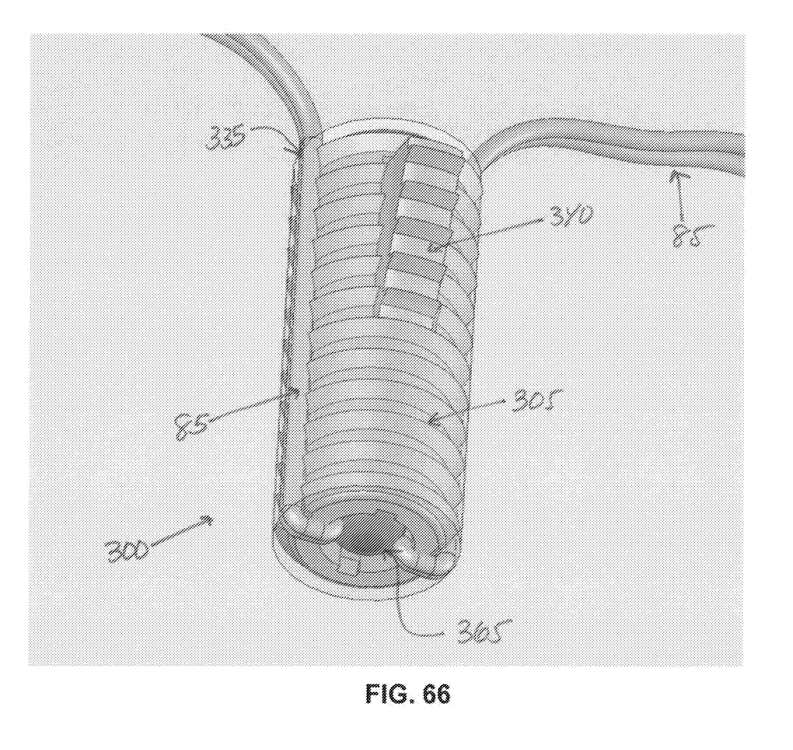

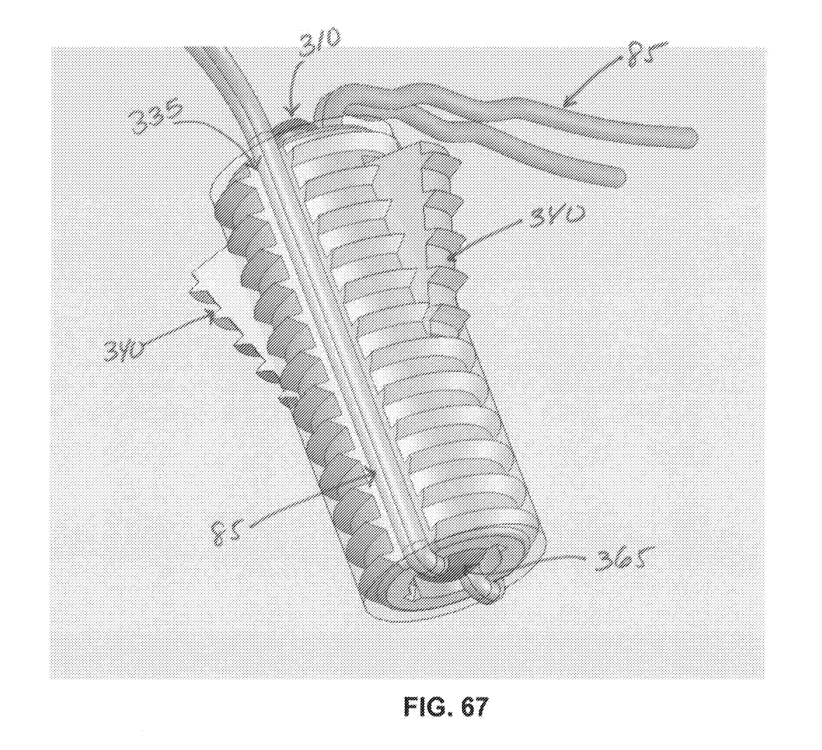

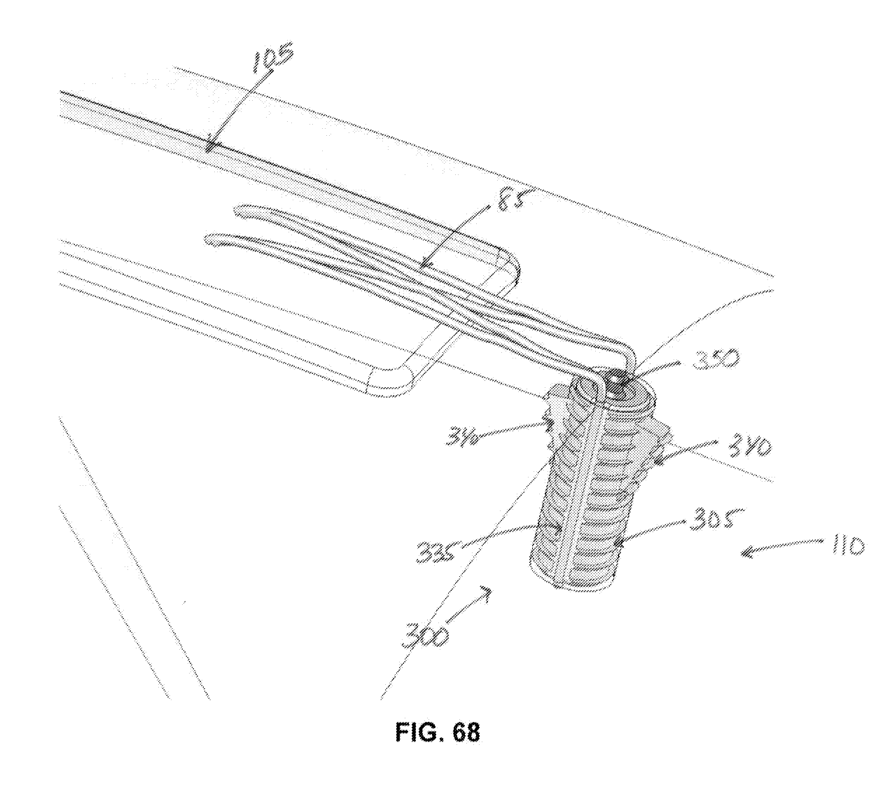

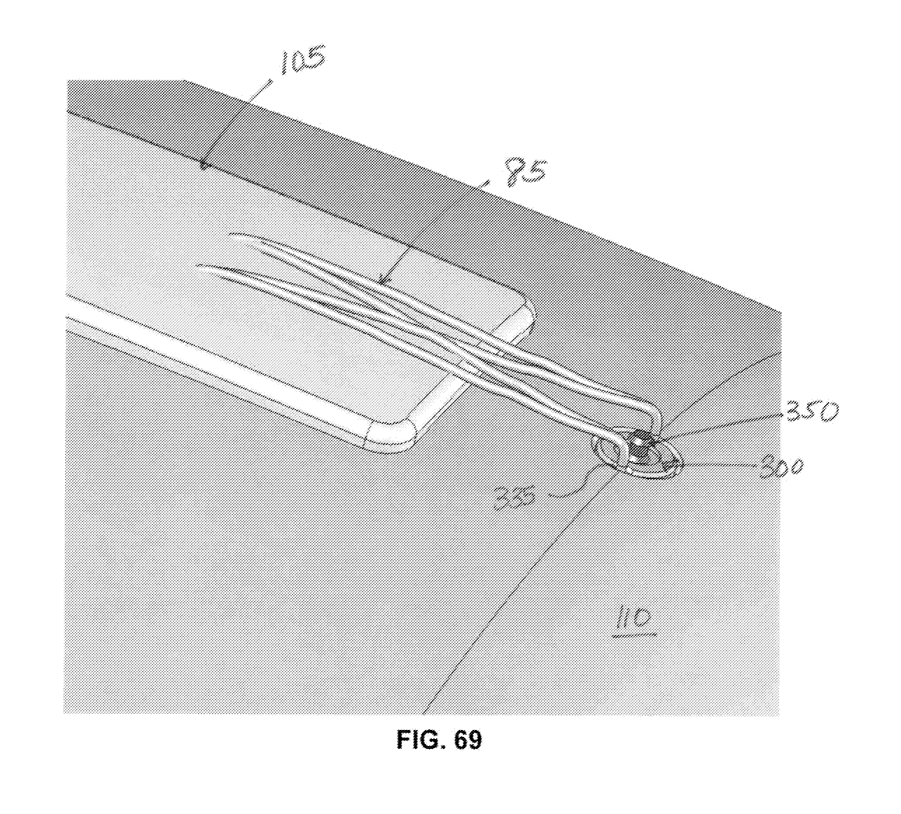

After elongated body 305 has been seated in bone hole 95, and looking now at FIGS. 63-69, shaft 345 of actuator 310 is moved from its second, intermediate position to its third, retracted position (i.e., the position shown in FIGS. 45-49), whereby to securely captured suture 85 between J-hook 365 and distal end surface 315 of elongated body 305, and radially expand wings 340 into the surrounding bone, so that elongated body 305 is secured to the bone, and suture 85 is secured to elongated body 305, whereby to capture suture 85 (and hence object 105) to the bone.

In this way object 105 may be secured to bone 110 without the necessity of tying knots in suture 85.

### Use of an Actuator with a J-Hook to Selectively Grapple/Slidingly Capture/Bind the Suture to the Suture Anchor, without Requiring the Actuator to Expand the Elongated Body of the Suture Anchor

It is also possible to use an actuator with a J-hook to selectively grapple/slidingly capture/bind the suture to the suture anchor, without requiring the actuator to expand the elongated body of the suture anchor.

Thus, in another form of the invention, elongated body 10 may be diametrically expandable independently of longitudinal movement of actuator 15 within elongated body 10. By way of example but not limitation, elongated body 10 may be formed out of a resilient material and the elongated body 10 may compress as it enters a bone hole, and thereafter expand within the bone hole, so as to bind the elongated body to the surrounding bone. Or elongated body 10 may otherwise bind or fixate itself within the bone hole. Thus, in this form of the invention, movement of actuator 15 within elongated body 10 is still used to selectively grapple/slidingly capture/bind the suture, but movement of actuator 15 is not used to diametrically expand elongated body 10.

### “Winged Body” Construction with Suture Capture Mechanism

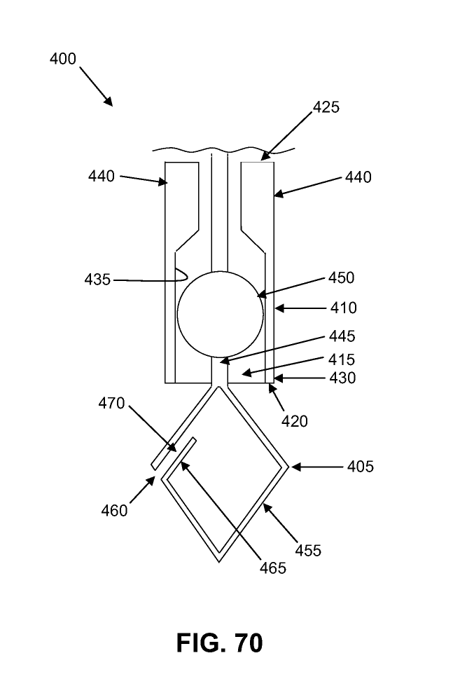

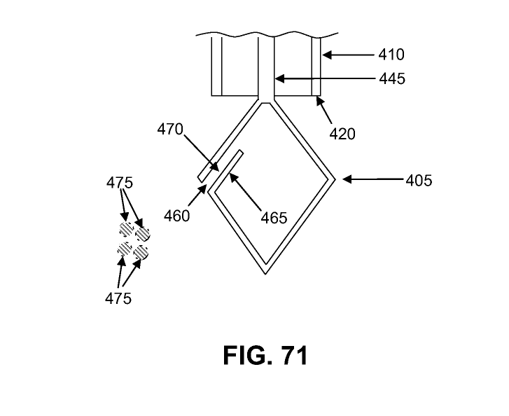

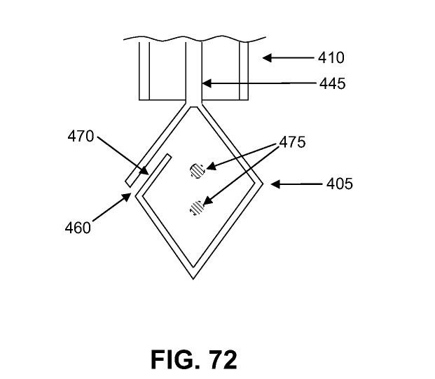

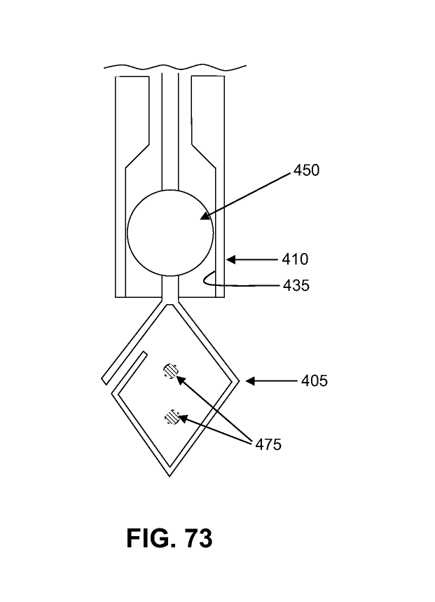

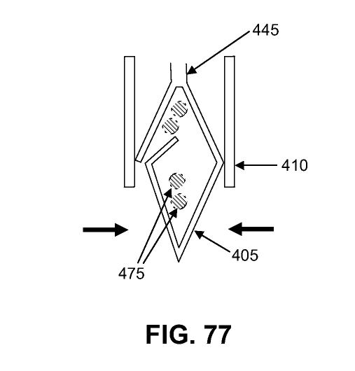

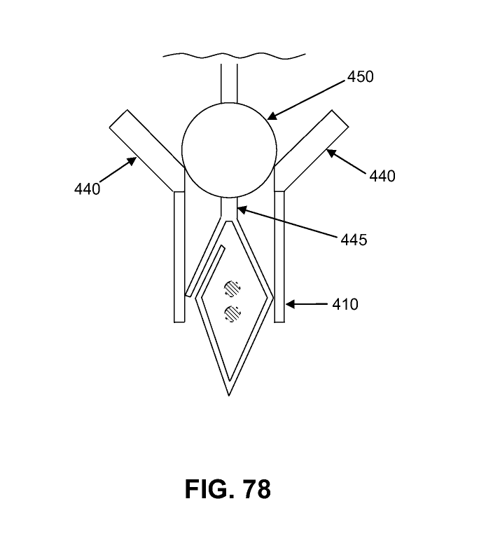

Looking now at FIGS. 70-78, there is shown a novel suture anchor 400 also formed in accordance with the present invention. Suture anchor 400 is generally similar to the suture anchor 200 disclosed above, except that in this form of the invention, J-hook 265 of suture anchor 200 is replaced with a suture capture mechanism 405, as will hereinafter be discussed in further detail.

More particularly, suture anchor 400 generally comprises an elongated body 410, and an actuator 415 slidably disposed therein. Suture capture mechanism 405 is disposed at the distal end of actuator 415.

Elongated body 410 comprises a distal surface 420, a proximal surface 425 and an outer surface 430. A bore 435 extends between distal surface 420 and proximal surface 425. A pair of diametrically-opposed wings 440 are hingedly connected to elongated body 410 so that the wings may selectively project outboard of outer surface 430 of the elongated body, whereby to bind the elongated body in a bone hole.

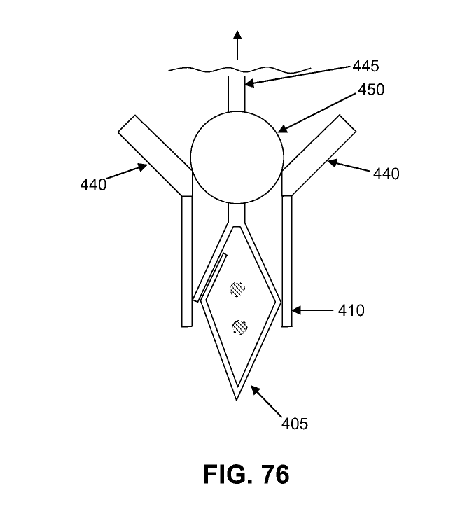

Actuator 415 generally comprises an elongated shaft 445 having at least one protrusion 450 disposed intermediate the elongated shaft. Protrusion 450 is positioned on elongated shaft 445 so that the protrusion is located distal to wings 440 when actuator 415 is in its first, extended position (FIGS. 70-73). Protrusion 450 is configured so that it will engage wings 440 when actuator 415 is moved proximally (i.e., when actuator 415 is retracted) into its aforementioned third, retracted position (FIGS. 76-78), whereby to deploy wings 440 outboard of elongated body 410 (and hence secure the suture anchor in a bone hole).

Actuator 415 terminates, at its distal end, in the suture capture mechanism 405. Suture capture mechanism 405 comprises a flexible, generally diamond-shaped structure 455 having an opening 460 formed therein. Preferably opening 460 is formed at one of the side apexes of diamond-shaped structure 455. In one preferred form of the invention, the diamond-shaped structure includes a fifth leg 465 so that opening 460 is in the form of a channel 470.

The flexible, generally diamond-shaped structure 455 is sized so that, at its widest point, it has a width which is greater than the diameter of bore 435 at the distal end of elongated body 405.

As a result of this construction, when actuator 410 is in its first, extended position (FIGS. 70-73), suture capture mechanism 405 is spaced from the distal end of elongated body 410, exposing opening 460. In this position, one or more strands of suture 475 can enter opening 460, preferably by passing down channel 470, so that the strands of suture will reside within the interior of the flexible, diamond-shaped structure 455.

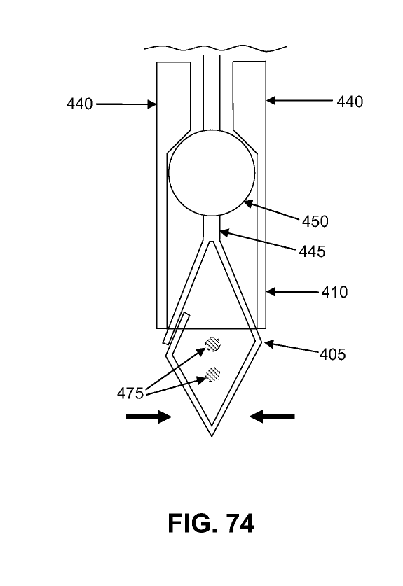

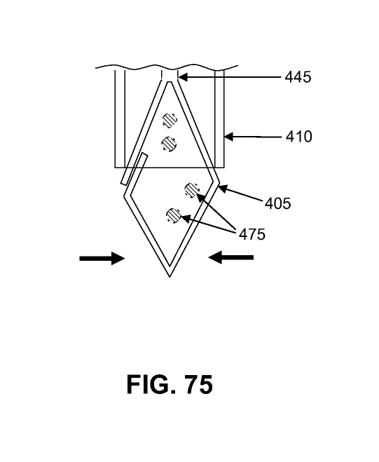

However, after one or more strands of suture have entered the interior of the flexible, diamond-shaped structure 455, actuator 415 can be moved proximally (i.e., retracted) from its first, extended position into its second, intermediate position (FIGS. 74 and 75). Inasmuch as the flexible, diamond-shaped structure 455 is wider than the diameter of bore 435, such proximal retraction of actuator 415 will force the flexible, diamond-shaped structure 455 to “partially collapse”, so that opening 460 closes down, slidably capturing the one or more strands of suture within the interior of the flexible, diamond-shaped structure 455. Thus, when actuator 415 is retracted into its second, intermediate position, the flexible, diamond-shaped structure 455 is partially collapsed, closing off opening 460, such that suture can slide within the flexible, diamond-shaped structure 455 but cannot escape from the interior of the flexible diamond-shaped structure.

Thereafter, when actuator 415 is retracted into its third, fully-retracted position (FIGS. 76-78), protrusion 450 moves proximally, so that wings 440 are deployed outboard. At the same time, the flexible, diamond-shaped structure 455 is drawn into the undersized bore 435 so that the flexible, diamond-shaped structure 455 is “fully collapsed” within the interior of elongated body 410, with suture strands 475 being securely bound within suture anchor 400 by this action.

The present invention is preferably used as follows:

(i) suture 475 is first threaded into suture capture mechanism 405 of suture anchor 400 (e.g., by passing the suture through opening 460) while actuator 415 is in its first, fully extended position;

(ii) actuator 415 is thereafter moved proximally into its second, intermediate position, so that suture strands 475 are slidably captured to suture anchor 400;

(iii) next, suture anchor 400 is pushed into a bone hole—as this occurs, suture strands 475 slide relative to the suture anchor but cannot escape from the interior of the flexible, diamond-shaped structure 455; and

(iv) actuator 410 is then retracted into its third, retacted position, so that wings 440 bind the suture anchor to the walls of the bone hole while the suture capture mechanism 445 further collapses and binds the one or more strands of suture to elongated body 405 of the suture anchor.

In one preferred form of the invention, opening 460 is sized so that it is slightly larger than the diameter of the suture, whereby to allow the suture to pass through opening 460—however, in this form of the invention, opening 460 is preferably only slightly larger than the diameter of the suture, so as to make it unlikely that the suture will pass back out of suture capture mechanism 405 through opening 460.

In another preferred form of the invention, a spring-biased gate selectively closes off the opening 460 formed in suture capture mechanism 405, so as to provide further assurances that the one or more strands of suture are properly captured to the suture anchor. This may be accomplished by forming fifth leg 465 so that it normally positively engages (or nearly positively engages) the adjacent length of the diamond-shaped structure, thereby effectively closing off channel 470, with fifth leg 465 (or adjacent portions of diamond-shaped structure 455 supporting fifth leg 465) giving way when a suture is forced down channel 460 (whereby to admit suture into the interior of the diamond-shaped structure 455).

In the foregoing description, suture capture mechanism 405 is discussed in the context of suture anchor 400, which uses a “winged body” construction (i.e., suture anchor 400 includes wings 440). However, it should be appreciated that suture capture mechanism 405 may also be used with an anchor body which does not include wings 440. By way of example but not limitation, suture capture mechanism 405 may be used with a resilient anchor body, or with any other anchor body which may be secured in bone.

### Modifications

It will be understood that many changes in the details, materials, steps and arrangements of parts, which have been herein described and illustrated in order to explain the nature of the invention, may be made by those skilled in the art without departing from the principles and scope of the present invention.

What is claimed is:

1. A suture anchor for anchoring a length of suture to bone, the suture anchor comprising:
an elongated body having a distal end surface, a proximal end surface, an exterior sidewall extending between the distal end surface and the proximal end surface, and an interior passageway extending between the distal end surface and the proximal end surface; and
an actuator comprising a distal end, a proximal end and a shaft extending between the distal end and the proximal end, the distal end of the actuator comprising a suture capture mechanism, the proximal end being configured for engagement by a handle, and the shaft being slidably disposed within the passageway;
the elongated body and the actuator being configured so that:
(i) when the actuator is in a first, extended position relative to the elongated body, the suture capture mechanism is spaced a sufficient distance from the distal end surface of the elongated body such that a length of suture may enter the suture capture mechanism;
(ii) when the actuator is in a second, intermediate position, the suture capture mechanism is positioned relative to the distal end surface of the elongated body such that the suture is slidably captured within the suture capture mechanism of the elongated body; and
(iii) when the actuator is in a third, retracted position relative to the elongated body, the suture capture mechanism is positioned relative to the distal end surface of the elongated body such that the suture is securely captured within the suture capture mechanism of the elongated body.

2. A suture anchor of claim 1 wherein the elongated body and the actuator include a control mechanism such that the actuator can be selectively held in one of its first, second and third positions.

3. A suture anchor of claim 2 wherein the control mechanism comprises a detent mechanism for releasably holding the actuator in one of its first, second and third positions.

4. A suture anchor of claim 3 wherein the control mechanism comprises three detents.

5. A suture anchor according to claim 1 wherein the elongated body comprises features on its exterior sidewall for engaging bone.

6. A suture anchor according to claim 5 wherein the features comprise ribs.

7. A suture anchor according to claim 1 wherein the elongated body has a first cross-sectional profile when the actuator is in its first, extended position and its second, intermediate position, and wherein the elongated body has a second, expanded cross-sectional profile when the actuator is in its third, retracted position.

8. A suture anchor according to claim 7 wherein the first cross-sectional profile of the elongated body is no greater than the cross-sectional profile of a hole in bone, and the second cross-sectional profile is greater than the cross-sectional profile of a hole in bone.

9. A suture anchor according to claim 8 wherein the elongated body comprises at least one wing, and further wherein the at least one wing is deployed outboard of the elongated body when the actuator is in its third, retracted position.

10. A suture anchor according to claim 1 wherein the elongated body is resilient.

11. A suture anchor according to claim 1 wherein the suture capture mechanism is a diamond-shaped structure configured to provide an opening into the interior thereof.

12. A suture anchor according to claim 11 wherein the opening is in the form of a channel.

13. A suture anchor according to claim 12 wherein the diamond-shaped structure comprises a fifth leg and the opening extends alongside the fifth leg.

14. A suture anchor according to claim 12 wherein the opening exists in the absence of any force being applied to the diamond-shaped structure.

15. A suture anchor according to claim 12 wherein the opening is provided as a result of a force being applied to the diamond-shaped structure.

16. A suture anchor according to claim 15 wherein the force is provided by engagement between the suture and the diamond-shaped structure.

17. A suture anchor according to claim 11 wherein the diamond-shaped structure has an open configuration when the actuator is in its first, extended position and the diamond-shaped structure has a partially collapsed configuration when the actuator is in its second, intermediate position, and wherein the diamond-shaped structure has a fully collapsed configuration when the actuator is in its third, retracted position.

18. A method for securing a suture to a bone, comprising:
providing a suture anchor comprising an elongated body and a suture capture mechanism slidably disposed relative to the elongated body:
positioning the suture capture mechanism so that it is spaced from the elongated body;
passing a suture into the suture capture mechanism;
repositioning the suture capture mechanism relative to the elongated body so that the suture is slidably captured within the suture capture mechanism of the elongated body;
positioning the elongated body in a bone hole; and
repositioning the suture capture mechanism relative to the elongated body so that the suture is bound within the suture capture mechanism of the elongated body, with the elongated body being secured in the bone hole.

19. A method according to claim 18 wherein the suture anchor comprises:
an elongated body having a distal end surface, a proximal end surface, an exterior sidewall extending between the distal end surface and the proximal end surface, and an interior passageway extending between the distal end surface and the proximal end surface; and
an actuator comprising a distal end, a proximal end and a shaft extending between the distal end and the proximal end, the distal end of the actuator comprising the suture capture mechanism, the proximal end being configured for engagement by a handle, and the shaft being slidably disposed within the passageway;
the elongated body and the actuator being configured so that:
(i) when the actuator is in a first, extended position relative to the elongated body, the suture capture mechanism is spaced a sufficient distance from the distal end surface of the elongated body such that a length of suture may enter the suture capture mechanism;
(ii) when the actuator is in a second, intermediate position, the suture capture mechanism is positioned relative to the distal end surface of the elongated body such that the suture is slidably captured between the suture capture mechanism and the elongated body; and
(iii) when the actuator is in a third, retracted position relative to the elongated body, the suture capture mechanism is positioned relative to the distal end surface of the elongated body such that the suture is securely captured between the suture capture mechanism and the elongated body.

20. A method according to claim 18 wherein the suture capture mechanism is a diamond-shaped structure configured to provide an opening into the interior thereof.

21. A method according to claim 20 wherein the opening is in the form of a channel.

22. A method according to claim 20 wherein the diamond-shaped structure comprises a fifth leg and the opening extends alongside the fifth leg.

23. A method according to claim 20 wherein the opening exists in the absence of any force being applied to the diamond-shaped structure.

24. A method according to claim 20 wherein the opening is provided as a result of a force being applied to the diamond-shaped structure.

25. A method according to claim 24 wherein the force is provided by engagement between the suture and the diamond-shaped structure.

26. A suture anchor for anchoring a length of suture to bone, the suture anchor comprising:
an elongated expandable body having a distal end surface, a proximal end surface, an exterior sidewall extending between the distal end surface and the proximal end surface, and an interior passageway extending between the distal end surface and the proximal end surface; and
an actuator comprising a distal end, a proximal end and a shaft extending between the distal end and the proximal end, the distal end of the actuator comprising a suture capture mechanism movably disposed relative to the elongated body;
wherein the suture capture mechanism comprises a diamond-shaped structure configured to provide an opening into the interior thereof.

27. A method for anchoring a length of suture to bone, the method comprising:
providing a suture anchor comprising:
an elongated expandable body having a distal end surface, a proximal end surface, an exterior sidewall extending between the distal end surface and the proximal end surface, and an interior passageway extending between the distal end surface and the proximal end surface; and
an actuator comprising a distal end, a proximal end and a shaft extending between the distal end and the proximal end, the distal end of the actuator comprising a suture capture mechanism movably disposed relative to the elongated body;
wherein the suture capture mechanism comprises a diamond-shaped structure configured to provide an opening into the interior thereof;
passing a suture into the suture capture mechanism;
repositioning the suture capture mechanism relative to the elongated body.
